(19) United States
(12) Patent Application Publication
(10) Pub. No.: US 2008/0194928 A1
(43) Pub. Date: Aug. 14, 2008

(54) SYSTEM, DEVICE, AND METHOD FOR DERMAL IMAGING

(76) Inventors: Jadran Bandic, Pancevo (YU); Djuro Koruga, Belgrade (YU); Rahul Mehendale, Jersey City, NJ (US); Sava Marinkovich, Chicago, IL (US)

Correspondence Address: STRATEGIC PATENTS P.C.., C/O PORTFOLIOIP, P.O. BOX 52050, MINNEAPOLIS, MN, 55402, US

(21) Appl. No.: 11/970,448

(22) Filed: Jan. 7, 2008

Related U.S. Application Data
(60) Provisional application No. 60/883,769, filed on Jan. 5, 2007.
(60) Provisional application No. 60/883,764, filed on Jan. 5, 2007.
(60) Provisional application No. 60/883,768, filed on Jan. 5, 2007.

(51) Int. Cl. A61B 5/00

(52) U.S. Cl. 600/306

(57) ABSTRACT

In embodiments of the present invention, systems and methods of a non-invasive imaging device may comprise an illumination source comprising an incident light source to direct light upon skin, and a detector for detecting the degree of polarization of light reflected from the skin. A system and method of determining a skin state may be based on an aspect of the polarization of the reflected light.

### CROSS-REFERENCE TO RELATED APPLICATIONS

This application claims the benefit of the following provisional applications, each of which is hereby incorporated by reference in its entirety: U.S. Patent Application Ser. No. 60/883,769, filed Jan. 5, 2007, “ALGORITHM TO ASSIST SKIN EXAMINATION”; U.S. Patent Application Ser. No. 60/883,764, filed Jan. 5, 2007, “REMOTE SKIN EXAMINATION”; and U.S. Patent Application Ser. No. 60/883,768, filed Jan. 5, 2007, “HOME USER DEVICE FOR SKIN EXAMINATION”.

### BACKGROUND

1. Field

The invention relates to methods and apparatus for enabling the collection of dermal and non-dermal images using a non-invasive imaging device, the development of a skin state 158 based at least in part on analysis of such images, and the monitoring of the skin state 158 by, at least, a collection and analysis of subsequent images.

2. Description of the Related Art

Skin is the largest organ of the integumentary system which further includes skin's accessory structures, such as hair, nails, scales, feathers, sweat glands and their products. Skin comprises multiple layers of epithelial tissues that guard underlying muscles and organs. Since skin is subject to constant attack from various external and internal factors, it can be afflicted by numerous ailments. Thus, it is critical to monitor skin health and the effect of any treatments, skin care products, or cosmetics applied to the skin.

While various methods exist for determining and monitoring skin health, most require access to a dermatologist or a dermatological facility. With a capacity shortage in dermatology, there may be difficulty, inconvenience, and prohibitively high cost in accessing the necessary resources. There is a need for a simple solution for skin health determination and monitoring that may be operable by an untrained or trained user, and, in the absence of an in-person consultation, where dermal images may be submitted to an expert, an analysis facility, or for automated analysis.

### SUMMARY

In aspects of the invention, an imaging device permits a user to take high magnification pictures of the skin in the vicinity of an area of concern and submit those pictures, optionally along with textual and data responses, for medical, non-medical, and cosmetic analysis, diagnosis and treatment recommendation and follow-up

In an aspect of the invention, a method and system of a non-invasive imaging device may comprise an illumination source comprising an incident light source to direct light upon skin; and a detector for detecting the degree of polarization of light reflected from the skin. In the method and system, the illumination source may be positioned to direct light at a selected angle alpha. Varying alpha may vary the depth of the measurement of the layers in the skin. Each depth may have a specific angle which produces a full polarized reflection. In the method and system, the incident light source may be an unpolarized light source. The unpolarized light may be white light, multiple selected wavelengths, or a single wavelength. The method and system may further comprise a sensor for capturing an image of the reflected light. The method and system may further comprise an optical facility for detecting reflected light from the skin. The method and system may further comprise a communication facility for transmitting the detected information. The method and system may further comprise a storage facility for storing information collected by the device.

In an aspect of the invention, a method and system for determining a skin state may comprise illuminating skin with an incident light source, detecting the degree of polarization of light reflected from the skin, and determining a skin state based on an aspect of the polarization of the reflected light. In the method and system, the incident light may be directed at a selected angle alpha. Varying alpha may vary the depth of the measurement of the layers in the skin. Each depth may have a specific angle which produces a full polarized reflection. In the method and system, the incident light source may be an unpolarized light source. The unpolarized light may be white light, multiple selected wavelengths, or a single wavelength. In the method of claim, the aspect of the polarization may be at least one of an orientation, an amplitude, a phase, an angle, a shape, a degree, an amount, and the like. In the method and system, determining may be done using an algorithm. The algorithm may involve artificial neural networks, fuzzy logic, fractal and multi-fractal analysis, and the like. The methods and systems may further comprise filtering the reflected light to obtain polarized light of at least one wavelength defined by the filter output. The algorithmic analysis may be performed on the filtered image. In the method and system, determining may involve creating an image from the difference between the reflected diffusion light and the reflected polarized light. In the method and system, determining may involve comparing the aspect of the polarization of the reflected light to a calibration signal. In the method and system, determining may further comprise considering at least one of user input and a visual analysis.

In an aspect of the invention, a non-invasive imaging device may comprise an illumination source comprising an incident light source to direct light upon an area of concern and a detector for detecting the degree of polarization of light reflected from the area of concern. In the method and system, the illumination source may be positioned to direct light at a selected angle alpha. Varying alpha may vary the depth of the measurement of the layers in the skin. Each depth may have a specific angle which produces a full polarized reflection. In the method and system, the incident light source may be an unpolarized light source. The unpolarized light may be white light, multiple selected wavelengths, or a single wavelength. The method and system may further comprise a sensor for capturing an image of the reflected light. The method and system may further comprise an optical facility for detecting reflected light from the skin. The method and system may further comprise a communication facility for transmitting the detected information. The method and system may further comprise a storage facility for storing information collected by the device.

In an aspect of the invention, a method of determining moisture levels in the skin may comprise emitting incident light towards a skin structure, detecting a degree of polarization and diffusion of the light reflected by the skin structure, and determining a moisture level based on the amount of polarized and diffused reflected light. The method and system may further comprise combining the assessment of moisture level with skin color measurements to determine luminosity. In the method and system, the incident light may be unpolarized light. The unpolarized light may be white light, multiple selected wavelengths, or a single wavelength. In the method and system, determining may involve use of an algorithm. In the method and system, determining a moisture level may be based on the ratio of polarized and diffuse light.

In an aspect of the invention, a method and system of determining elasticity of the skin may comprise emitting incident light towards a skin structure, detecting an aspect of polarization of the light reflected by the skin structure, correlating the aspect of polarization with a concentration of elastin, and determining elasticity level based on the concentration of elastin. In the method and system, determining may involve use of an algorithm. In the method and system, the incident light may be unpolarized light. The unpolarized light may be white light, light of multiple selected wavelengths, or a single wavelength of light.

In an aspect of the invention, a method and system of determining firmness of the skin may comprise emitting incident light towards a skin structure, detecting an aspect of polarization of the light reflected by the skin structure, correlating the aspect of polarization with a concentration of at least one of an elastin, a collagen, and an activity of a sebaceous gland, and determining firmness based on the concentration of at least one of elastin and collagen and sebaceous gland activity. In the method and system, the sebaceous gland activity may be indicated by at least one of a number of glands, percent of glands open/closed, and level of clog/fill. In the method and system, correlating may involve use of an algorithm.

In an aspect of the invention, a method and system for obtaining dermal biophysical properties may comprise performing a spectral analysis of image data acquired from the degree of polarization of reflections of incident light from skin structures, wherein the property is at least one of a structure, form, concentration, number, size, state, and stage of at least one of a: melanocyte, melanin, hemoglobin, porphyrin, keratin, carotene, collagen, elastin, sebum, sebaceous gland activity, pore (sweat and sebaceous), moisture level, elasticity, luminosity, firmness, fine line, wrinkle count and stage, pore size, percent of open pores, skin elasticity, skin tension line, spot, skin color, psoriasis, allergy, red area, general skin disorder or infection, tumor, sunburn, rash, scratch, pimple, acne, insect bite, itch, bleeding, injury, inflammation, photodamage, pigmentation, tone, tattoo, percent burn/burn classification, mole (naevi, nevus), aspect of a skin lesion (structure, color, dimensions/asymmetry), melanoma, dermally observed disorder, cutaneous lesion, cellulite, boil, blistering disease, congenital dermal syndrome, (sub)-cutaneous mycoses, melasma, vascular condition, rosacea, spider vein, texture, skin ulcer, wound healing, post-operative tracking, melanocytic lesion, non-melanocytic lesion, basal cell carcinoma, seborrhoic keratosis, sebum (oiliness), nail- and/or hair-related concern, and the like.

In an aspect of the invention, a method and system for determining a skin state may comprise obtaining the answers to a series of subjective questions regarding the skin, obtaining an objective skin analysis using a dermal imaging device, and combining the subjective and objective results algorithmically to obtain a skin state.

In an aspect of the invention, a system and method for providing recommendations for skin care based on a skin state and a skin care goal may comprise obtaining a skin state of an individual, categorizing the individual by skin state, and recommending products and regimens that are effective for other individuals of the category in achieving the skin care goal. In the method and system, the system may be operable over a network. In the method and system, the skin state may be determined based on analysis of the degree of polarization of light reflected from the skin of the individual.

In an aspect of the invention, a method for tracking the effectiveness of a skin care product or regimen may comprise obtaining a baseline skin state assessment, recommending a monitoring interval based on at least one of the skin care goal, product, and regimen, obtaining a second skin state assessment, comparing the second assessment to the baseline assessment to determine progress towards a skin care goal, and optionally, optimizing the regimen or product in order to improve a skin state. In the method and system, the skin assessment may be based on analysis of the degree of polarization of light reflected from the skin of the individual.

In an aspect of the invention, a personalized skin condition analysis system and related methods may comprise an imaging device, comprising an illumination source comprising an incident light source to direct light upon skin, and a detector for detecting the degree of polarization of light reflected from the skin, and a user interface for controlling the device. In the methods and system, the device may be adapted to interact with a physical interface to download image data to update a record of at least one of a practitioner, a spa, a salon, cosmetic sales, a cosmetics manufacturer, a clinical trials database, and a third party database. In the method and system, the illumination source may be positioned to direct light at a selected angle alpha. Varying alpha may vary the depth of the measurement of the layers in the skin. Each depth may have a specific angle which produces a full polarized reflection. In the method and system, the incident light source may be an unpolarized light source. The unpolarized light may be white light, multiple selected wavelengths, or a single wavelength. The method and system may further comprise a sensor for capturing an image of the reflected light. The method and system may further comprise an optical facility for detecting reflected light from the skin. The method and system may further comprise a communication facility for transmitting the detected information. The method and system may further comprise a storage facility for storing information collected by the device.

In an aspect of the invention, a non-invasive imaging device may comprise an illumination source comprising an incident light source to direct light upon skin; and a detector for detecting a characteristic of the light reflected from the skin. In the device, the illumination source may be positioned to direct light at a selected angle alpha. Varying alpha may vary the depth of the measurement of the layers in the skin. Each depth may have a specific angle which produces a full polarized reflection. In the device, the incident light source may be a polarized light source or unpolarized light source. The unpolarized light may be at least one of white light, light of a single wavelength, and light of multiple single wavelengths. The device may further comprise a sensor for capturing an image of the reflected light. The device may further comprise an optical facility for detecting reflected light from the skin. The device may further comprise a communication facility for transmitting the detected information. The device may further comprise a storage facility for storing information collected by the device. In the device, the reflected light may be at least one of polarized light and unpolarized light.

In an aspect of the invention, a method and system for determining a skin state may comprise illuminating skin with an incident light source; detecting a characteristic of the light reflected from the skin; and determining a skin state based on at least one characteristic of the reflected light. In the method and system, the incident light may be directed at a selected angle alpha. Varying alpha may vary the depth of the measurement of the layers in the skin. Each depth may have a specific angle which produces a full polarized reflection. In the method and system, the incident light may be unpolarized or polarized light. The unpolarized light may be at least one of white light, light of a single wavelength, and light of multiple single wavelengths. In the method and system, the reflected light may be at least one of polarized light and unpolarized light. In the method and system, the characteristic may be at least one of light source, light intensity, wavelength of light, angle of light, electrical and magnetic properties of the light, and polarization state of the light. An aspect of the polarization may be at least one of an orientation, an amplitude, a phase, an angle, a shape, a degree, and an amount. In the method and system, determining may be done using an algorithm. The algorithm may involve artificial neural networks, fuzzy logic, or fractal and multi-fractal analysis. The method and system may further comprise filtering the reflected light to obtain light of a wavelength defined by the filter output. The algorithmic analysis may be performed on the filtered image. In the method and system, determining may involve creating an image of the difference between reflected diffusion light and reflected polarized light. In the method and system, determining may involve comparing the aspect of the polarization of the reflected light to a calibration signal. In the method and system, determining may further comprise considering at least one of user input and a visual analysis.

In an aspect of the invention, a non-invasive imaging device may comprise an illumination source comprising an incident light source to direct light upon an area of concern; and a detector for detecting a characteristic of the light reflected from the area of concern. In the device, the illumination source may be positioned to direct light at a selected angle alpha. Varying alpha may vary the depth of the measurement of the layers in the skin. Each depth may have a specific angle which produces a full polarized reflection. In the device, the incident light source may be a polarized light source or unpolarized light source. The unpolarized light may be at least one of white light, light of a single wavelength, and light of multiple single wavelengths. The device may further comprise a sensor for capturing an image of the reflected light. The device may further comprise an optical facility for detecting reflected light from the skin. The device may further comprise a communication facility for transmitting the detected information. The device may further comprise a storage facility for storing information collected by the device. In the device, the reflected light may be at least one of polarized light and unpolarized light.

These and other systems, methods, objects, features, and advantages of the present invention will be apparent to those skilled in the art from the following detailed description of the preferred embodiment and the drawings. All documents mentioned herein are hereby incorporated in their entirety by reference.

### BRIEF DESCRIPTION OF THE FIGURES

The invention and the following detailed description of certain embodiments thereof may be understood by reference to the following figures:

FIG. 1 depicts a skin care system for skin health analysis and monitoring, and skin care assessment and recommendation.

FIG. 2 depicts a mechanism for light polarization by a skin structure.

FIG. 3 depicts a process for skin care examination.

FIGS. 4A & B depict a front and back view of a dermal imaging device.

FIG. 5 depicts a skin health monitoring page of a skin care system.

FIG. 6 depicts an interactive modeling tool of a skin care system.

FIG. 7 depicts a recommendations page of a skin care system.

FIG. 8 depicts a user interface of a skin care system.

FIG. 9 depicts a welcome page of a skin care system.

FIG. 10 depicts a questionnaire page of a skin care system.

FIG. 11 depicts a skin image capture page of a skin care system.

FIG. 12 depicts a results page with bar graphs of a skin care system.

FIG. 13 depicts a results page with line graphs of a skin care system.

FIG. 14 depicts a summary screen of a skin care system.

FIG. 15 depicts an elasticity summary screen of a skin care system.

### DETAILED DESCRIPTION

Provided herein may be methods, systems, and a device for dermal and non-dermal imaging. Throughout this disclosure the phrase “such as” means “such as and without limitation”. Throughout this disclosure the phrase “for example” means “for example and without limitation”. Throughout this disclosure the phrase “in an example” means “in an example and without limitation”. Throughout this disclosure, the term “product” refers to any medical, non-medical, cosmetic, skin, hair, or nail care product. Generally, any and all examples may be provided for the purpose of illustration and not limitation.

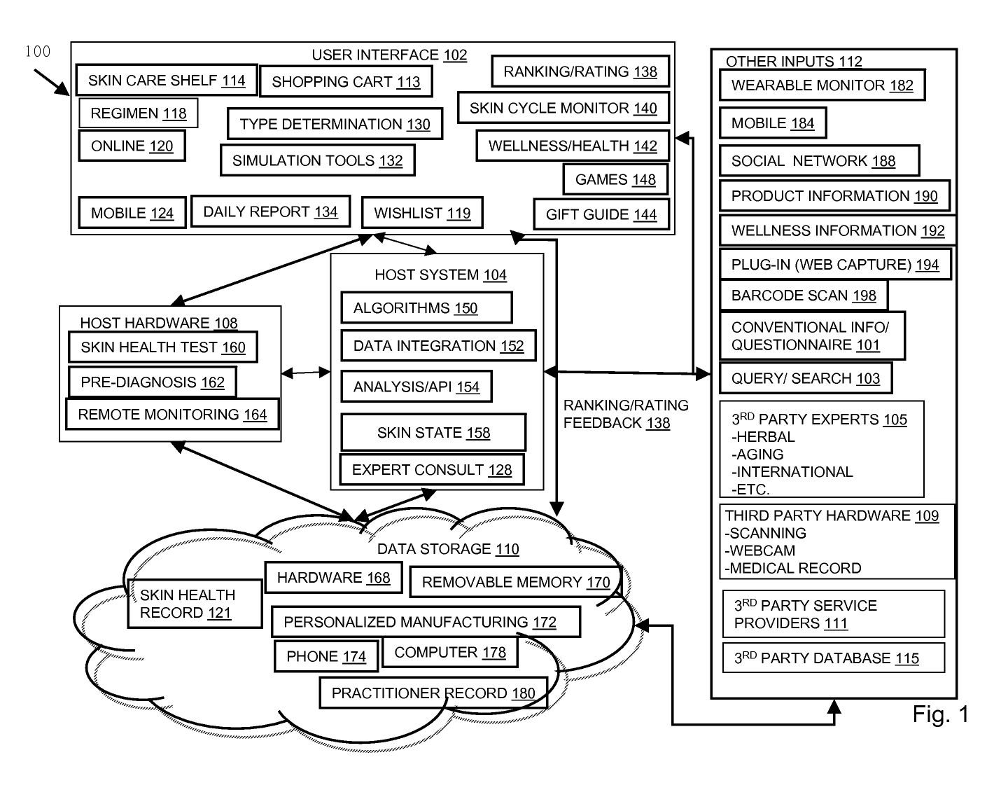

Referring to FIG. 1, a system for skin health analysis, monitoring, and recommendation may comprise host hardware 108, such as an imaging device 108, for capturing biophysical skin properties such as in a skin health test 160, performing pre-diagnosis 162, and performing remote monitoring 164; a user interface 102 interfacing with the host hardware 108, an online platform 120, or a mobile platform 124 for capturing demographic information, additional anecdotal information on skin health, current skin care regimen 118, rankings and ratings 138 of current skin care products and regimen, populating a skin care shelf 114, and accessing a skin cycle monitor 140, health and/or wellness information 142, games 148, a gift guide 144, a wishlist 119, a Daily Report 134, simulation tools 132, a type determination engine 130, a shopping cart 113, and the like; a host system 104 for processing and analyzing captured information such as by employing an algorithm 150, obtaining an expert consultation 128, data integration 152, and analysis tools/API's 154 to define a skin state 158; other inputs 112 to the host system 104, which may be subject to ranking/rating feedback 138, for providing additional granularity in identifying, monitoring, and adjusting a skin state 158, such as a wearable monitor 182, a mobile communications device 184, a social network 188, product information 190, wellness information 192, a plug-in (web capture) 194, a barcode scan 198, conventional information/questionnaire answers 101, a query/search 103, third part experts 105, third party hardware 109, third part service providers 111, and the like; and data storage 110 for storing data from the host hardware 108, host system 104, user interface 102, and other inputs 112, such as hardware 168, removable memory 170, a wireless communication device 174, a computer 178, a practitioner record 180 such as a dermatologist, general physician, aesthetician, spa employee, salon employee, cosmetic salesperson, and the like, a personalized manufacturing record 172, and the like. While dermal embodiments are contemplated throughout this disclosure, except where context prohibits such embodiments should be understood to encompass non-dermal embodiments, such as and without limitation any hair, nail, agricultural, veterinary, and internal embodiments, as well.

An imaging device 108 may be used to capture images of skin structures to obtain biophysical skin properties such as in a skin health test 160, a pre-diagnosis 162, remote monitoring 164, and the like. The imaging device 108 may also be adapted to capture images of non-dermal structures, such as hair, nails, teeth, eyes, internal organs and structures, and the like. The imaging device 108 may use an internal or external light source to provide a specific sequence of irradiation using unpolarized light, such as diffusion light, white light, monochromatic light, light of multiple single wavelengths, and the like, then polarized light in order to obtain data on skin structures. In embodiments, the incident light may be polarized or unpolarized) and the reflected light may be polarized or unpolarized. The polarized light may result from the reflection on the skin and is not polarized from the light source. The capture and storage of the reflections enables the imaging and analysis of skin lesions, as well as all types of skin diseases, skin problems, and cosmetic concerns and indications. Analysis of polarized reflections may enable obtaining thermal, electrical, and magnetic properties of the imaged skin area. The images may be transmitted to an analysis facility 154, analyst, practitioner and the like, which may also include assessment with patient questionnaires, to determine a final analysis of skin health. The device 108 may also employ specific targeted wavelengths, such as in the red, green, and blue areas, to identify key features, based on spectroscopic and quantitative analysis of skin lesions. In embodiment, the device 108 may be adapted to emit polarized light. The device 108 may be adapted to emit more than one type of light and may be able to switch among or combine various light sources. The skin health analysis may be compared with a previous user skin health analysis, other users' skin health analysis, other users' experience data, and ingredient, product, and regimen characteristics to provide a recommendation for and track the effectiveness of a product or regimen 118.

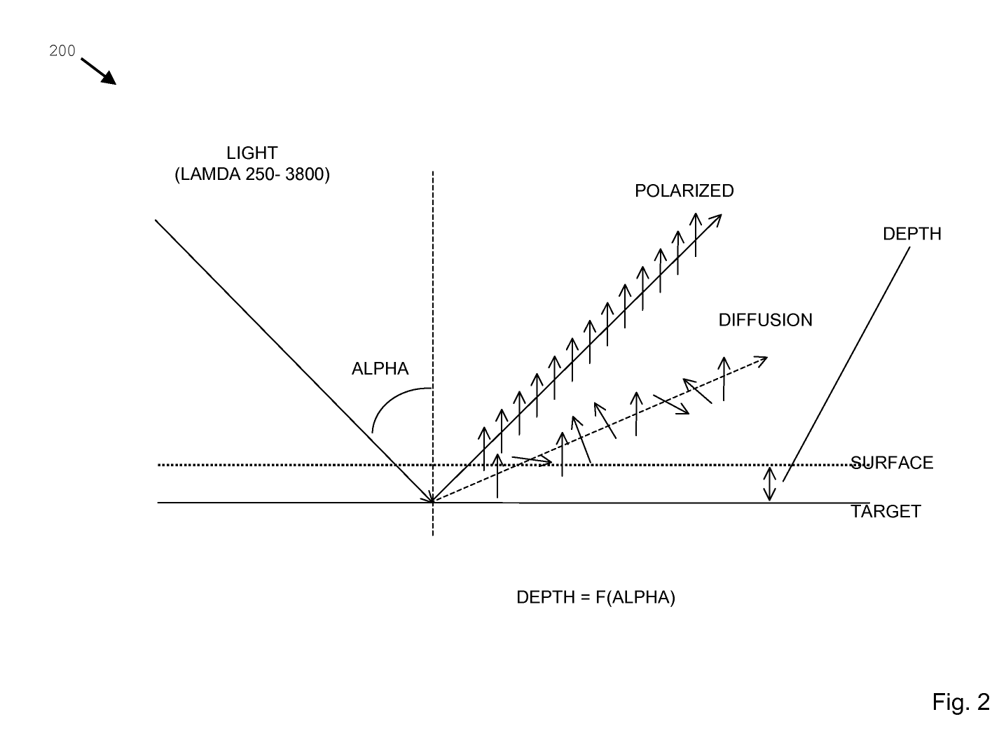

Referring now to FIG. 2, in an embodiment, the imaging device 108 may comprise an illumination source to direct unpolarized light, diffusion light, white light, monochromatic light, light of multiple single wavelengths, polarized light, and the like, upon the skin at an angle alpha, a sensor for detecting reflected light from a skin structure, and an image storage device for storing and transmitting the captured images. A skin structure may be at least one of a cell, a molecule, a group of cells, a group of molecules, an epidermis and sublayers, a basement membrane, a dermis, a subcutis, a gland, a stratum, a follicle, a pore, a vascular component, and the like resident within the skin. In an embodiment, the light source may be white light for generating reflected light, such as reflected polarized light and/or diffusion light, to measure the electrical and magnetic components of the skin. White light may be emitted as a combination of wavelengths of light across the spectrum of visible light. Incident unpolarized light may be directed at its target at a defined angle ‘alpha’ from vertical. As the value of alpha changes, such as and without limitation over a range of 45 to 65 degrees from vertical, incident unpolarized light may interact with different structural elements of the skin since varying the angle of incidence affects the depth of penetration. The angle alpha may be changed by changing the position of the light source, either manually, through a remote control, through a user interface 102, and the like. The relationship between depth of penetration and alpha may be defined by the formula depth=f(alpha). For each skin structure which may correspond to a particular known depth within the skin, there may be a specific angle of incidence which produces a full polarized reflection. By analyzing the reflected light, either polarized and/or diffusion, information on the underlying skin structures responsible for the reflection may be obtained. The polarization of the light may be due to classical/quantum effects of skin structures interacting water. That is, skin structures possess enough of a magnetic and electric field to be able to alter the polarization of light as it strikes the structures and to affect the wavelength of light as it strikes the structures. An aspect of the polarization of the reflected light, such as an orientation, an amplitude, a phase, an angle, a shape, a degree, an amount, and the like, may correlate with various measures associated with the particular skin structures targeted, and ultimately, a skin state 158. For example, a lesion present in a particular skin structure may cause the diffusion of a portion of the reflected light resulting in reflected light that is partially polarized and partially diffused. For example, collagen structures are one indicator of a biological difference between a benign and a malignant melanocytic skin lesion. The collagenous differences may affect the polarization state of reflected light, and the resultant images may indicate locations of tumor center and tumor periphery. Such images may aid a practitioner in visualizing excision margins. Because melanocytes are located at the lower part of the epidermis, the appropriate wavelength may be selected for this depth as well as for the chromophores within the various types of nevi.

If incident light is polarized, only the electrical properties of skin will be apparent but unpolarized incident light may reveal both the electrical and magnetic properties of skin. While using polarized light may generate improved induction of optical activity, the data sets generated may be of less value as compared to the data sets captured using incident unpolarized light, such as white light, a monochromatic light, light of multiple single wavelengths, and the like. By measuring the effects between 10⁻³⁴ and 10⁻³⁰ Js, one can make measurements at the border area of quantum and classical physics effects on the skin and as a difference of action of electrical and magnetic forces of valence electrons of skin's biomolecules.

In an embodiment, the wavelength and/or intensity of the incident light may be modified in order to measure the presence of specific molecules, such as collagen, elastin, cadherin, hemoglobin, and the like. Certain molecules possess the property of endogenous fluorescence. For example, if incident light is limited to a particular wavelength, such as 325 nm, collagen may be detected at an emission wavelength of 400 nm and 405 nm. Table 1 lists certain illustrative examples of excitation and emission maxima of biological molecules that exhibit endogenous fluorescence, such as amino acids, structural proteins, enzymes and coenzymes, vitamins and vitamin derivates, lipids, porphyrins, and the like. To detect the presence of specific molecules in the skin, a user may shine a light of a specified wavelength, such as and without limitation those shown in the excitation maxima column, onto the skin and collect reflected light to identify the presence of specific emission wavelengths in the reflections. It may be understood by one knowledgeable in the art that many different single wavelengths and combinations of wavelengths of light may be used to illuminate the skin.

| | | | Emission |
|---|---|---|---|
| | | Excitation | maxima |
| Endogenous fluorescence | | maxima (nm) | (nm) |
| Amino acids | Tryptophan | 280 | 350 |
| | Tyrosine | 275 | 300 |
| | Phenylalanine | 260 | 280 |
| Structural proteins | Collagen | 325 | 400, 405 |
| | Elastin | 290, 325 | 340, 400 |
| Enzymes and coenzymes | FAD, flavins | 450 | 535 |
| | NADH | 290, 351 | 440, 460 |
| | NADPH | 336 | 464 |
| Vitamins | Vitamin A | 327 | 510 |
| | Vitamin K | 335 | 480 |
| | Vitamin D | 390 | 480 |
| Vitamin B6 compounds | Pyridoxine | 332, 340 | 400 |
| | Pyridoxamine | 335 | 400 |
| | Pyridoxal | 330 | 385 |
| | Pyridoxic acid | 315 | 425 |
| | Pyridoxal | 330 | 400 |
| | 50-phosphate | | |
| | Vitamin B12 | 275 | 305 |
| Lipids | Phospholipids | 436 | 540, 560 |
| | Lipofuscin | 340-395 | 540, 430-460 |
| | Ceroid | 340-395 | 430-460, 540 |
| Porphyrins | | 400-450 | 630, 690 |

FAD, flavin adenine dinucleotide;
NADH, reduced nicotinamide adenine dinucleotide;
AND(P)H, reduced nicotinamide adenine dinucleotide phosphate.

In an embodiment, light may be emitted at any wavelength, such as across the range from 280 nm to 3800 nm. Incident light may be blue, yellow, orange, red, or some other light.

Continuing to refer to FIG. 1, in an embodiment, the light source may be integral to the device 108 or provided from an associated source. The light source may be a light-emitting or laser diode (LED) of any wavelength, such as and without limitation 280, 340, 360, 385, 405, or 480 nm incident excitation wavelengths. Wavelengths in the ultraviolet and infrared ranges may also be emitted by the device 108. The light source may be diffusion light, white light, monochromatic light, light of multiple single wavelengths, incandescent, electroluminescent, fluorescent, halogen, ultraviolet, polarized light, collimated light, light provided by a wireless communications device, light provided by a fiber optic cable, and the like. In an embodiment, the light source may comprise a diffuser to provide diffuse incident light.

In an embodiment, a sensor for detecting reflected light from the skin may be embodied in optics resident in a CCD camera, CMOS-based imaging system, digital camera, webcam, camera embedded in a communications device such as a cell phone or iPhone, PDA (Personal Digital Assistant), a watch or other wearable device for continuous monitoring of the skin as in a sports-type indication, a third party device 109, a scanner, and the like. The sensor may be adapted to absorb any wavelength of light, such as near IR or visible wavelengths. The sensor may be adapted to automatically filter out particular wavelengths. The sensor may be adapted to image any size area, such as a small portion of the skin, the full face, a complete cutaneous examination, and the like. The sensor may be adapted to operate without any intervening fluids between the device 108 and the area of concern, or may be used with an oil-like application or other reflective media to the area of concern. The sensor may be adapted to detect reflected light, from any distance from the area or when in contact with the area of concern, which may be used for subsequent visual and/or algorithmic analysis. The images generated from this reflected light may be considered both visual as well as spectroscopic images or electromagnetic skin maps. The sensor may have an internal calibration scale that enables measuring the size of the region being imaged as well as the distance from the imaged area. In an embodiment, a lens may focus the reflected light from the detection optics onto a visible-NIR sensitive CCD, CMOS, or other sensory device. In an embodiment, the sensor may be adapted to acquire images at a high frame rate. In an embodiment, the device may possess a high magnification lens.

In an embodiment, the device 108 may store captured images for analysis and/or transmittal to an analysis facility 154. The analysis facility 154 may be a practitioner, an automated analysis tool, a practitioner employing analysis tools, and the like. Data storage 110 may occur manually when image capture is initiated, may occur automatically upon contact with the skin, may be remotely controlled, and the like. Data may be stored in an internal device memory 168 or may be stored externally in memory media 170 such as USB memory, an external hard drive, a mass storage device, and the like. The device may be able to connect externally, either through a wired connection or wirelessly, to a computer, such as a laptop, kiosk, desktop computer, central server, and the like. For example, the connection may be a direct USB connection. When the device 108 is connected to the computer, captured data may be downloaded or transmitted either automatically or upon manual initiation from the device 108 to the computer. For example, the device 108 may have a cradle in connection with a computer. When the device 108 is placed in the cradle, data may be transmitted or downloaded from the device 108. Additionally, the device 108 may receive software updates when connected to the computer, such as through the cradle. In embodiments, the device 108 may have no internal storage and may only be able to transmit or store data externally through a persistent hard-wired or wireless connection. Data transmittal and storage may be a fully automated process or may be manually operated. Data may be transmitted over a wireless network connection, a cellular connection, a wired connection, a Bluetooth connection, and the like. Data transmittal from the device 108 may enable remote assessment techniques. In an embodiment, non-image data may also be stored and/or transmitted by the device 108 as described herein, such as voice responses, text responses, video data, and the like. The device 108 may have an internal microphone to record audio, a video camera to record video, a keyboard input to record text responses, and the like. In an embodiment, the device 108 may use externally available audio and video.

In an embodiment, data storage may be in a skin health record 121. The skin health record 121 may be an object or database or repository for an individual that contains information on key medical, non-medical, and cosmetic indications related to a user's skin. This may comprise images, graphics, icons, written history, personal demographic information, levels of cosmetic conditions such as moisture, elasticity, firmness, texture, color level, or non-medical conditions such as inflammation, and the like. A user may self-populate the record 121 with data from any device 108, 109 or input 112. The record 121 may contain a history of skin concerns, comments, a user blog, and the like. In an embodiment, the skin health record 121 may auto-populate upon acquisition of an image. For example, when a user submits their first image for analysis, a record 121 may be automatically created and populated with information, which may be edited, derived from the image and its analysis.

In an embodiment, data storage 110 may occur in a practitioner record 180. A practitioner record 180 may be a repository of key health characteristics including background demographic data, personal information, information on diet, skin health record 121 and the like. It may have embedded images, links to other image data files, tracking effectiveness of personal skin products, medical products, and OTC products and the like and their historical impact on key parameters. It may also capture community data or data of selected individuals who may be similar to the patient or user and may include rankings and comments and the like

In an embodiment, data storage 110 may be in a personalized manufacturing record 172. Based on the skin health measurement 160, product ingredients to obtain a desired effect to make the skin healthy may be selected. This ingredient selection may be achieved by analyzing and tracking the change of various skin health parameters through the application of various products and ingredients through using the device 108 and tracking the change of the skin health over time through a personalized manufacturing record 172. Once the selected product ingredients are identified, they may be mixed to create a product best suited for the individual's skin characteristics and/or desired goals (such as improved moisturization). Thus a personalized product may be developed for the user. Additionally, this same process could be used for creation of specific customized skin products and ingredients for medical and non-medical purposes and conditions.

In an embodiment, the form of the data captured may be compatible with any standard image processing and manipulation software and techniques, word processing software, slideshow presentation, spreadsheet applications, and the like. For example, the captured data may be in any suitable image format, such as jpeg, tiff, pict, png, bmp, gif, pdf, and the like. In an embodiment, multiple images may be captured as a movie or a movie may be constructed from combining multiple images.

In an embodiment, the device 108 may be powered by any suitable source, such as an electric power plug, a battery, solar power, USB power, and the like. A user may initiate power to the device 108 in order to begin acquiring images. Acquisition may commence automatically, may commence when the device 108 is placed against the skin, may commence when a trigger, such as a button, is actuated by a user, and the like.

The device 108 may have a display for viewing the area to be imaged. For example, a user may use the display with positioning tools to obtain exact images over time, such as a series of images taken over different days. The display may be integral to the device 108 or may be a separate display. For example, the device 108 may be connected to a monitor, such as that of a computer, using a wired connection or a wireless connection. In an embodiment, a user interface 102 to the device 108 may display a real time view of the imaging.

In an embodiment, the device 108 may have security features in order to protect the privacy of user data. For example, the device 108 may have a unique MacID with encryption technology.

In an embodiment, the device 108 may be associated with peripherals or other functional attachments. For example, the device 108 may be associated with a blood pressure monitor or sensor, a heart rate monitor or sensor, and the like. For example, the device 108 may be used to perform a pre-diagnosis 162 of a skin lesion while also monitoring other endpoints such as blood pressure, heart rate, and the like in order to assess other aspects of health in addition to skin health.

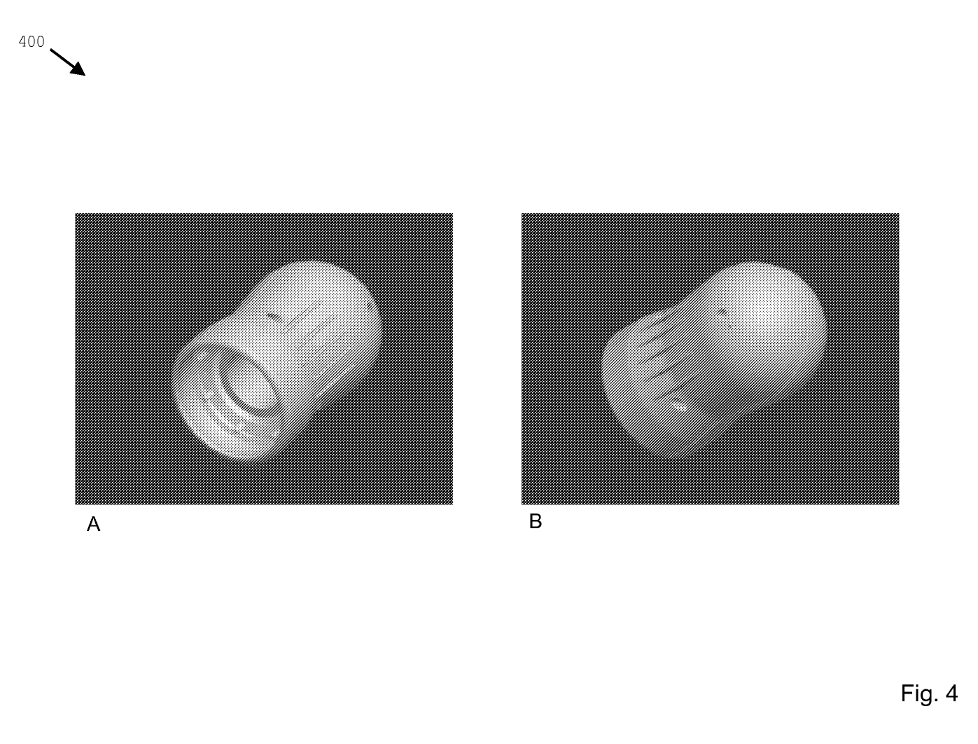

In an embodiment, the device 108 may be sized to permit a user to operate the device 108 in a handheld fashion. The device 108 may sized for portability. The device 108 may adapted for single-handed operation. For example, the device may be embodied as in FIGS. 4 A & B, but it may have multiple other embodiments in any shape and/or size, such as a mirror, a large device adapted to image a large area, a PDA, a scanner, a mobile communication device, and the like. In FIG. 4 A, the illumination source is visible as a ring of LED's around a central detection area. In both images, the size, handheld nature, and portability are clearly demonstrated. The ease of operation enables even an inexperienced user, such as a home user connected to a laptop, to employ the device 108. The device 108 may be a self-contained unit and not part of a larger camera system. In an embodiment, the device 108 may be designed for one handed ergonomic holding. In an embodiment, the device 108 may be used with or without application of reflective media. In an embodiment, the device 108 may be used to capture images at a distance, close-up, in direct contact, and the like. For example, software loaded on a computer interfaced with the device 108 may prompt for near distance and far distance image capture.

In an embodiment, the device 108 may also be a standalone, non-hand-held version, which may be used to take images or particular body components or materials.

In an embodiment, the device 108 may be adapted for use as a component of a minimally invasive medical device associated with laparoscopy, cytoscopy, ureteroscopy, arthroscopy, endoscopy, dermoscopy, gynecology, urology, dentistry, natural orifice insertion analysis such as through ears, mouth, anus, nose, and external breast cancer analysis through the skin, and the like. For example, the system may be able to process the data and to appear on a video monitor or other display in a surgical suite or other medical setting. A medical professional may be able to select a viewing mode, such as still image capture or video capture, and may be able to manually adjust the parameters of the light source, sensor and display to assist in observation, identification, and monitoring with the device 108. In an embodiment, the system may be pre-programmed with various protocols for the various types of medical procedures and tissues types that a medical professional may encounter such that the system may automatically handle the device 108 based on the medical professional's indication of the type of procedure and tissue being examined.

In an embodiment, the device 108 may enable a skin health test 160. The imaging device 108 may be used to perform a skin health test 160 to learn the characteristics of the skin and to obtain a diagnosis. The hardware device may capture an image and enable analysis of the image. The imaging components within the device 108 may enable measuring various skin health characteristics like color, age, damage, collagen, elastin, pores and types, keratin, and the like. The skin health test 160 may be performed in the home, in a spa, clinic, hospital, from a mobile phone at any location, and the like. The skin health test 160 may be used in conjunction with specific background information through questionnaires, image upload, genetic testing, DNA samples, and lifestyle habits to determine a skin state 158. The test 160 would respond with specific information related to the biophysical health of the skin, a portion of which would be physical and genetic disposition to certain medical or non-medical or cosmetic problems or conditions.

In an embodiment, the device 108 may enable a pre-diagnosis 162. This is a system of pre-diagnosis where a practitioner (such as the user, a dermatologist, medical practitioner, aesthetician, and the like) may receive or request from a user to take an image and/questionnaire of a skin concern or the like and receive a pre-diagnosis based on algorithmic analysis of pre-existing conditions. The user may submit a questionnaire and image with a pre-diagnosis of conditions prior to going to see a practitioner and allow a follow-up. Images captured by the device may be submitted to obtain a preliminary diagnosis to enable effectively referring the case to the best practitioner. The pre-diagnosis 162 may be performed by software algorithms on the images, manual analysis, a combination thereof, and the like. The pre-diagnosis 162 may include the preliminary assessment as well as indicate the time required and the steps required for the final diagnosis or assessment. This pre-diagnosis 162 feature may enable effective scheduling of the practitioner. The pre-diagnosis 162 could also help screen for particular skin issues as well as identify users with certain issues.

In an embodiment, the device 108 may enable remote monitoring 164. The user may use the device in the privacy of their home, work, or any other location to perform remote monitoring 164 and submit images to track progress of their skin's health or medical conditions. A practitioner may be able to remotely guide changes in treatment or guide on prevention factors. Remote diagnosis may greatly increase efficiency of progress monitoring since users will not have to make a physician trip to the provider, and the provider could conveniently select a time during the day to observe the patients change. The monitored data may be viewed as a recording or in real time.

In an aspect of the invention, the imaging device 108 may illuminate an area of concern at a known angle of incidence with unpolarized light. To obtain a spectroscopic image of the magnetic properties of the area only, the reflected polarized light, which possesses the electrical properties of the area of concern, may be subtracted from any reflected diffusion light, which possesses electromagnetic properties of the area of concern. The distribution of pixels in the image corresponding to the diffusion light and reflected polarized light may be determined and indicated by any conventional means. For a known image sensor, a one-to-one mapping of pixel image distribution between the diffusion light image, corresponding to an electromagnetic signal, and reflected polarized light, corresponding to an electrical signal image, may be made with a distribution of the intensity of the spectroscopic data for the same area. A magnetic gradient image of the area may be made by equipment such as an AFM-MMR (Atomic Force Microscopy in Magnetic Mode Regime) and from the one-to-one correspondence, a skin state 158 may be based on the gradient image, diffusion light image, and reflected polarized light image.

In an embodiment, the device 108 may be an imaging device 108 for performing digital spectroscopic imaging of the skin. Incident unpolarized light may be delivered, either vertically or on an angle alpha from vertical, from an unpolarized light source associated with the device 108, such as a white light, diffuse light, monochromatic light, light of multiple single wavelengths, and the like, to a target skin structure. White light, which possesses both electrical and magnetic properties, when incident onto a skin structure at a particular angle interacts with the structure's components and leads to the reflected light having a polarized light component. In embodiments, the incident light may be polarized. Unpolarized light reflected by skin structures may become polarized, at least in part. The reflected light, either polarized or diffusion light, may be captured by the device 108. Such multispectral skin imaging may be used to develop an electromagnetic skin topography. By measuring aspects of the polarization of the reflected light such as an orientation, an amplitude, a phase, an angle, a shape, a degree, and an amount, and the wavelength of the reflected light, the biophysical properties of skin structures may be obtained. A skin state 158 may be determined from the aggregate biophysical data obtained from one or more skin structures as well as a visual analysis of the captured images and any additional data obtained from the user anecdotally. For example, the skin state 158 may encompass data on moisture, wrinkles, pores, elasticity, luminosity, and any of a number of measures, as described herein. By varying alpha, the angle of incident white light, the depth of penetration of the light to skin structures may be varied. Each depth within the skin corresponds to different skin structures. For each skin structure or depth, there may be a specific angle which produces a full polarized reflection. For example, a certain angle of incidence may be used to obtain data for skin structures within the epidermis, however, the angle of incidence may need to be changed in order to obtain data on skin structures within the subcutis which resides at a different depth within the skin. The angle of incidence may be modified to penetrate the skin anywhere from a few microns up to a few centimeters, thus enabling the capture of reflections from other non-dermal structures. For example, the device 108 may be used as a non-invasive imaging tool, such as to image tumors, breast cancer, melanoma, and the like. In an embodiment, the area to be imaged may be any biological tissue that may have normal or pathologic variations in its structure, such as variations in the tissue's birefringent properties. For example, scars, keloids, hypertrophic scars, and stria all have organizations of collagen fibers that are different from normal skin. Since collagen is a primary determinant of cutaneous wound repair, it may be of interest to monitor changes in collagen structure and concentration. For example, the stage of healing may be determined by the size of collagen bundles which may increase as healing progresses, by the organization of collagen structures at the molecular or small-fibril level which may increase as healing progresses, by the return or increase of birefringence, and the like. Since collagen structures are polarization-sensitive, changes that occur in the structures may be monitored using a polarization-based technique during scar formation, the healing process, and treatment of scars, as has been and will be further described herein.

Being able to measure the electrical and magnetic properties of various skin structures may enable the differentiation between healthy and non-healthy skin structures. Normal or healthy skin structures exhibit a unique conformation that differs from the conformation exhibited by equivalent structures when unhealthy or abnormal. These conformational changes can be detected by differences in an aspect of the light reflected off of skin, such as an aspect of the polarization of the reflected light. The aspect of polarization may be the wavelength of the light, an orientation, an amplitude, a phase, an angle, a shape, a degree, an amount of polarization of the light, and the like. According to Maxwell's equations, light can be described as comprising an electric field and a magnetic field which can be described as two vectors, E and B, which behave as waves. The vectors are perpendicular to the propagation direction of the light, and they are orthogonal to each other. Furthermore, given the electric field E, B can be determined via Maxwell's equations, and vice versa. Thus, by measuring the electrical component of the light reflected by the skin structures, the magnetic component or the degree of polarization/polarization state may be determined. By comparing those electrical and magnetic readings from the polarized component of reflected light and non-polarized white light to that of normal or healthy skin structures incident with light at the same or similar angles, changes may be detected in the skin structure and its molecular or structural conformation. Based on the amount or other aspect of both electrical and magnetic determination, specific defects such as cancer, skin diseases, cosmetic indications and the like, may be detected, since each range of measurements may correspond to a particular defective conformation. If any other molecules, cell, or structure are now incident with the same type of light at the same angle, the strength of certain wavelengths of the reflected component may enable the measurement of the intensity of the difference in conformation states of the measured component. The polarization state of the reflected light may be described by a number of parameters. The polarization state may be described in terms of the polarization ellipse, specifically its orientation and elongation. Parameters which may be used to describe the polarization state may include the azimuth angle (ψ) which is the angle between the major semi-axis of the ellipse and the x-axis, the ellipticity (ε) which is the ratio of the two semi-axes, the ellipticity angle which is the arctangent of the ellipticity, the eccentricity, the amplitude and phase of oscillations in two components of the electric field vector in the plane of polarization, and the like. For example, an ellipticity of zero corresponds to linear polarization and an ellipticity of 1 corresponds to circular polarization. The polarization of the reflected light may be at least one of elliptical, linear, circular, left-circular, right-circular and any potential combinations thereof.

In an embodiment, determining a skin state 158 may comprise processing and analyzing 154 the reflected light to obtain images for visual and spectroscopic analysis. Analysis 154 may be facilitated by examining the wavelength and other characteristics of the reflected light. For example, if the incident light is white light, the reflected light may be filtered to examine a collection of wavelengths or a single wavelength and, ultimately, a specific skin structure fluorescence. In another example, monochromatic or semi-monochromatic light, such as provided by an LED may be used to excite targeted fluorophores and chromophores. In this example, fluorescence of deeper layers may be extracted. The reflected light in this example may also be filtered to isolate a specific fluorescence. In another example, varying the wavelength of the illuminating light may enable detection of biophysical properties from various depths within the skin. In addition, certain chromophores, such as the various forms of hemoglobin found in blood, have specific absorption bands; thus processing of data created with different color light may yield information about chromophore distribution that may be polarization-sensitive. The wavelength dependence may be obtained in several ways: 1) illuminate sequentially with light of a single wavelength or multiple single wavelengths and collect each resultant image separately; or 2) illuminate with white light and examine the reflected light for individual wavelengths or a collection of individual wavelengths either during detection or during processing. Algorithms 150 may be used to obtain information from data obtained by either method by processing and analyzing one or more wavelengths of light to form a spectroscopic, polarization-based image. In an embodiment, the combination of both techniques may enable the elimination of the reflection from the surface of the skin.

In an embodiment, filtering may be employed to filter out a range of wavelengths, such as those belonging to the ultraviolet, infrared, near infrared, visible, and the like. The filter may be a digital or an analog filter. For example, captured images may be processed by software that may be able to employ digital filter techniques to process the images for analysis. For example, using software, any digital filter parameter may be selected such as a particular cutoff wavelength, a set of single wavelengths, a sampling interval, and the like. For example and without limitation, a digital filter may be used to isolate reflections of 405, 458, 488, 532, 580, and 633 nm wavelengths. In another example, an analog filter may be employed to filter the images as they are captured, such as a filter that is integral to the optics of the device 108, or as they are stored, transmitted, manipulated, processed, and the like, such as with an external analog filter. Filtering the images may result in obtaining images of underlying structures and/or a specific pattern of polarization. Filtering the images may result in the separation of the electrical and magnetic components of the reflected light. Filtered images may be subjected to algorithmic analysis. Filtering may eliminate reflections due to skin surface reflections by isolating specific wavelengths of light. For example, sebaceous glands may appear as bright spots in an image when only a certain wavelength of light is isolated for analysis, while isolation of a different wavelength of light enables the visualization of all the pores in the imaged area. Thus, the fluorescence from deeper layers may be isolated.

In an aspect of the invention, a host system 104 may comprise algorithms 150, data integration 152, analysis tools/API's 154, a skin state 158, an expert consult 128, and the like. The skin state 158 may be a data object or characterization of skin based on tests 160, pre-diagnoses 162, and monitoring 164 performed by a device 108, user input, expert consult 128, other inputs 112, analysis 154, algorithms 150, and the like. The skin state 158 along with all of the underlying data and user information may be stored in a skin health record 121. In an embodiment, the host system 104 may comprise server architecture. The host system may be technology agnostic. The host system 104 may comprise one or more cloud computing, service-oriented architecture, distributed objects, and the like.

In an embodiment, expert consult 128 may provide analysis, recommendations, assessment advice, and the like. The skin image data collected as well as the pre-diagnosis, in addition with any other allied data such as physician's diagnosis, insurance, blood analysis, and the like may be referred to an expert either by the user or a practitioner, or by other users to obtain an analysis, recommendation or assessment advice. Experts could be located in geographically distant locations, and may have very different skills. For example, the skin image data and analysis may be shared at the request of another user with an herbal specialist in India, or the user may request the image data to be shared with an aging expert in France to learn of best suited skin care treatment from their experience. The expert's consultation analysis may be maintained on the host system 104 as part of the skin history record 121 and may be accessed by the user at their convenience, or shared with other users.

In an embodiment, the system 104 may be a home-based, in clinical or medical settings, at spas and salons, at a cosmetics counter and in cosmetics sales, and the like to perform skin analysis discretely and accurately in a low cost, rapid, and secure fashion. In embodiments, the device 108 may integrate with a user interface 102, online platform 120, mobile platform 124 and the like to perform analysis 154, skin state 158 record keeping, obtain referrals/analysis from a remote practitioner or algorithm 150, and the like. The home-based system 104 may allow a practitioner, who may be any qualified or unqualified person to give advice, to analyze cosmetic or non-cosmetic conditions that may be captured by an imaging device 108 or third party device 109 and give advice and recommendations on products, regimen, diet, lifestyle and the like based on inputs from questionnaires, uploaded images, and the like. The system may consist of a starter website that may be customizable for a personal business where the practitioner could organize clients' cosmetic skin health, track their regimens, recommend products, be their online advisor, and the like. This would leverage the analysis and device platform to allow a practitioner to analyze comments, images, questions, and/or concerns and the like and give advice, consultation on lifestyle improvement and tracking. A spa/salon based system may enable personalized skin assets. For example, the spa may own the device, the device may capture images to feed a large scale display adapted to present a skin condition, and then a practitioner may be able to simulate the effect of treatment. Users may compare a skin state 158 with peers or other spa goers and generate recommendations based on what worked for them or what they bought. Desired improvements may be correlated to ingredients and most effective products/regimens 118 for the users' skin. The spa/salon based system 104 may generate product/service recommendations based on a skin state 158, offer one-click shopping based on recommendations and enable sku tracking, offer wellness packages such as through a contractual relationship, provide the ability to port regimen from spa to spa, from home to spa, and the like, enable optimization of regimens/advising such as helping practitioners tailor the length of a procedure, enable development of targeted therapies, enable clear, visual communication to clients, generate effectiveness of products/services reports, and the like. Reports may be based on or comprise correlation with other users, feedback on regimen 118, modifications of a regimen 118, skin cycle monitoring, and the like. A medical practitioner based system, such as a dermatologist, general physician, metabolist, and the like, may enable pre-diagnosis, may link to the practitioner's scheduling system, may enable pre-pricing of services, may enable follow-up tracking, and the like. A cosmetic sales or retail based system 104 may enable integration with inventory of product enabling clearing of inventory. A handheld/portable device 108 may be used at a makeup counter, in a drugstore, at a home or trade makeup show/party, and the like. Users may purchase peripherals/accessories for the device, such as a holster, charger, and the like. Users may pay-per-scan or may have a subscription scanning service and the like. The system 104 may be based in health clubs, gyms, resorts, and the like. A cosmetics manufacturing/testing based system may enable skin state-based product design, targeting skin care samples to particular consumers, and the like. The system 104 may be veterinarian based to monitor veterinary dermal- and non-dermal concerns. The system 104 may be based in a hospital, ER, military setting, and the like to enable rapid assessment of medical conditions, triaging urgent skin care, and the like. The system 104 may be agriculturally based to enable application to fruits, vegetables, and other such agricultural products. The system 104 may be used in a battlefield scenario or in an austere environment, such as in space flight, air flight, underwater, submarine, and the like, to enable wound management, battlefield diagnosis and triage, and the like. The system 104 may be research based to enable comparing any materials and their specific composition. Based on using the reading of the electrical property of the light, a user may be able to determine a similarity or difference between imaged material.

In an embodiment, determining a skin state 158 may comprise employing an analysis 154. In an embodiment, the acquired data may be analyzed by a practitioner, such as a physician, dermatologist, spa employee, clinical trial practitioner, aesthetician, cosmetologist, nutritionist, cosmetic salesperson, and the like. The practitioner may analyze the data upon acquisition, visually, with the assistance of an algorithm 150, expert consult 128, database 115, and the like. In an embodiment, the practitioner may be remote from the location of data acquisition. In an embodiment, an algorithm 150 may be used to process and analyze 154 the reflected light to obtain spectroscopic images, either automatically or under the control of a user, practitioner, and the like. For example, to obtain a spectroscopic image of the magnetic properties of the area only, an algorithm 150 may be used to generate an image of an area of concern using the difference between the reflected polarized light, which possesses the electrical properties of the area, and the reflected diffusion light, which possesses the electromagnetic properties of the area of concern. Algorithms 150 may be rules-based software and processes to 1) analyze imaging evidence to obtain skin health, 2) correlate skin health with ingredients, medicaments, and/or products that may be best suited for the determined skin health, 3) correlate skin health with peers in a skin health community, and 4) recommend and design personalized products based on skin health and/or other like users usage experience, 5) observe measurable changes in skin health, and the like. Algorithms 150 may be automated. Algorithms 150 may be used to analyze 154 medical concerns, such as degree of suspicion of cancer, rash analysis, and the like. Algorithms 150 may be used to analyze 154 non-medical concerns, such as the effectiveness of a medical, non-medical, or cosmetic regimen 118, a pimple avoidance regimen 118, a sun-protection effectiveness, an itch prevention cream, and the like. Algorithms 150 may be useful for correlating desired improvements with ingredients and most effective products for improving or maintaining the user's skin health. The algorithm 150 may utilize a calibration scale to determine the skin structures imaged based on the angle of incidence, wavelength and intensity of the light source, an aspect of the reflected light, filter parameters, and the like. Algorithms 150 may be useful for determining a dermascopic effect, a luminescence effect, a spectroscopic effect, and the like. For all algorithms 150, there may be an input, an output, and functional parameters to modulate the algorithm 150. In an embodiment, analysis 154 may comprise examining at least one of: physical data and/or an image of the material using diffusion white light; physical data and/or an image of material using light of a single wavelength or multiple single wavelengths; physical data and/or an image of the material using polarized, reflected light of a certain angle; physical data and/or an image of the material generated using the difference between diffusion white light and polarized reflected light of a certain angle; physical data and/or an image of the material generated using the difference between light of a single or multiple wavelengths and polarized, reflected light of a certain angle; and the like. Algorithms 150 may be used with data and images generated by the device 108 or third party hardware 109. Algorithms 150 may be used with data and mages captured using any image capture device or technique, employing any kind of incident light, such as unpolarized light, polarized light, monochromatic light, diffuse light, white light, multiple single wavelength light, and the like. In embodiments, any captured data or image may be subjected to algorithmic analysis, as described herein.

In an embodiment, the algorithm 150 may be based on artificial neural networks and fuzzy logic. For example, the algorithm 150 may be used in skin lesion diagnosis based on a probabilistic framework for classification. Two kinds of data may be inputs to the neural network: numerical data such as intensity, size, numbers, and the like, and descriptive data such as white, gray, dark, and the like. Fuzzy logic may directly encode structured descriptive data in a numerical framework. Based on associative memories, learning algorithms 150, and adaptive control system behavior, neural and fuzzy machine intelligence may enable correspondence between input data taken from collected images and a biophysical skin state 158.

In an embodiment, the algorithm 150 may be based on fractal and multi-fractal analysis of images based on biophysical and spatio-temporal data. Both digital image data and spectroscopic data of skin may be analyzed using Hausdorff dimensions (fractal property) and Kolmogorov's entropy (K-entropy). Then, spectroscopic data may be divided into spatio-temporal cells and analyzed as multi-fractal objects, yielding information about a level of functional disharmony of skin structures (epidermal and dermal). Structural data of these two analyses can be correlated to determinate a one-to-one correspondence between them. Once fractal correlations between digital image data and spectroscopic data of skin are established, it may be possible to obtain information about a functional state of skin structures through multi-fractal analysis of digital image data.

In an embodiment, an algorithm 150 may be for the analysis 154 of data integrity. For example, an algorithm 150 may be able to determine if the image has been captured in high enough detail to render subsequent analyses reliable.

In an embodiment, an algorithm 150 may be useful for the analysis of skin characteristics, obtaining the biophysical properties of the skin, and determining a skin state 158. The skin state 158 captures a combination of underlying skin structure with time-based variance. Some variation may be predictable but some may be based on a transient condition like infection, sunburn, hormonal imbalance, and the like. The algorithm 150 may be able to measure aspects such as the structure, form, concentration, number, size, state, stage, and the like of melanocytes/melanin, hemoglobin, porphyrin, keratin, carotene, collagen, elastin, sebum, sebaceous gland activity, pores (sweat and sebaceous), wrinkles, moisture, elasticity, luminosity, all forms of the aforementioned, such as derivatives, salts, complexes, and the like. The algorithm 150 may be used to make a quantitative assessment of clinical, medical, non-medical, and cosmetic indications, such as moisture level, firmness, fine lines, wrinkle count and stage, pore size, percent of open pores, skin elasticity, skin tension lines, spots, skin color, psoriasis, allergies, red areas, general skin disorders and infections, or other skin related concerns for the user such as tumors, sunburns, rashes, scratches, pimples, acne, insect bites, itches, bleeding, injury, inflammation, photodamage, pigmentation, tone, tattoos, percent burn/burn classification, moles (naevi, nevus), aspects of skin lesions (structure, color, dimensions/asymmetry), melanoma, dermally observed disorders and cutaneous lesions, cellulite, boils, blistering diseases, management of congenital dermal syndromes, (sub)-cutaneous mycoses, melasma, vascular conditions, rosacea, spider veins, texture, skin ulcers, wound healing, post-operative tracking, melanocytic lesions, non-melanocytic lesions, basal cell carcinoma, seborrhoic keratosis, sebum (oiliness), nail- and/or hair-related concerns, and the like. Either manually or as determined by an algorithm 150, a targeted wavelength or wavelengths may be employed for specific endpoint measurements. Either a specific wavelength or multiple wavelengths may be chosen for the incident light or a specific wavelength or wavelengths may be isolated by filtering, as described herein. An algorithm 150 may determine the presence, absence, structure, form, and the like of particular skin structures based on the properties of the reflected light. For example, an algorithm 150 may detect which axes/angle the light is polarized on and compare this to signature emission spectra of individual proteins/underlying skin structures. Each skin structure may have a unique signature pattern based on the electrical and magnetic contributions of molecule(s) present in the skin structure. The algorithms 150 may identify, analyze and separate the electrical and magnetic components of the unique polarization signal, as described herein. The signals may correlate with the aggregate conformation state of molecules in the skin structure. By comparing this signal to a standard calibration signal, aspects of the underlying skin structures may be determined. The standard calibration signal may be provided by a catalog of skin structures/molecules and their specific wavelength of observation. The catalog may be developed by the technique described herein or any other spectroscopic technique. For example, to determine moisture levels in the skin, an algorithm 150 may determine a ratio of the reflected polarized light and reflected diffusion light and correlate the ratio with a moisture level. Ideally, close to 100% polarized light may be generated from reflections, however if a portion of the reflected light is diffusion light, such as 95% polarized, 5% diffusion, the amount of diffused light may be correlated with a level of moisture. Incident unpolarized light may interact with a skin structure and lead to varying amounts of polarization of the reflected or refracted light. This polarized reflected or refracted light strength may be measured. This polarization may be as much as 100 percent, however, the reflected polarized strength may even be less than 100% in some cases. The incident angle and the imaged material would help determine the maximum strength possible for the polarization of the reflected light. It should be understood that there may be a maximum amount of polarization with a maximum of 100% for a particular incident angle, but any amount of polarization ranging from 0 to 100% polarized may be expected from the light reflected by any skin structure. The underlying cause for the differences in reflection may be due to the ratio of the captured and free water in the skin. To determine elasticity, an algorithm 150 may determine the concentration of elastin per area of concern. To determine luminosity, an algorithm 150 may combine moisture levels and skin color into a single, objective assessment. Objective measures may be correlated with an expert grading scale or other external measure. To determine firmness/tightness, an algorithm 150 may combine an assessment of collagen and elastin concentrations in an area of concern along with the activity of sebaceous glands (as measured by number of glands, percent open/closed, level of clog/fill). The algorithm 150 may be able to overlay varying wavelengths and intensities and spectroscopic techniques, such as reflectance, excitation/emission, and the like. The algorithm 150 may be able to process and analyze 154 images collected by the device 108 or any other imaging device using unpolarized light, polarized light, or a combination thereof. The algorithm 150 may be able to process and analyze 154 many different types of images, such as thermoelectromagnetic (TEM) images or electromagnetic (EM) images, images collected with incident polarized light, traditional dermoscopy images, spectroscopic images, conventional images, harmonized light images, and the like. The algorithm 150 may be able to calculate a variance measurement of skin state 158 over time. Determining a skin state 158 may also include, in addition to the processing and analysis of images of the skin for various measures and endpoints as described herein, a visual analysis of the images, user entered information, and third party information, such as lifestyle, smoking history, exercise habits, diet, allergies, and the like. For example, a user may enter anecdotal information, such as medication they may be taking, recent overexposure to sun, stage in a menstrual cycle, and the like.

In an embodiment, an algorithm 150 for determining a skin state 158 may facilitate measuring, tracking, and monitoring a skin state 158 as well as the effectiveness of a regimen 118, topical and/or systemic therapies, avoidance routines, diet, and the like. For example, the skin state 158 may be measured at intervals and current measurements may be compared to previous measurements to determine skin health changes. As will be further described herein, the results from the algorithm 150 may feed into a recommendation engine to provide feedback and modifications to aspects of the regimen 118.

In an embodiment, an algorithm 150 for determining a skin state 158 may enable a diagnosis. The diagnosis may be an early diagnosis by distinguishing between critical and non-critical indications. For example, the algorithm 150 may be able to distinguish between a minor sunburn and a third degree sunburn requiring medical attention. Use of the device 108 to capture images enables a user to readily transmit the images to any practitioner for remote assessment, to track progression of a skin condition, rapidly compare images to previous images, other user images or third party images, such as images in a dermascopic database 115, and the like, and to make an immediate assessment with no need for historical knowledge, and the like. Historical data and the results of modeling tools 132 may be compared to the images to assist in analysis, either by an algorithm 150, a practitioner, or a practitioner employing an algorithm. Also, in addition to images, user input in the form of audio, video, or text anecdotes describing the issue, such as a level of pain, a sensation of heat, an itchiness, and the like, may be useful in analyzing the images to determine a diagnosis. The algorithm 150 may enable principal component analysis (PCA), which may be a biomedical analysis used in conjunction with spectrometric analysis for analyzing medical and health conditions. The algorithm 150 may enable a simple pattern analysis for diagnosis. The algorithm 150 may be able to determine the thermo- and electroconductivity conditions of skin lesions. In an embodiment, the algorithm 150 may be able to diagnose a melanocytic lesion by examining the images for the relationship of changes in collagen and porphyrin, as a change in collagen but not porphyrin may indicate a change from a normal lesion to a dysplastic lesion. The skin state 158 may be compared with a table of indicators for various types of lesions. In an embodiment, the algorithm 150 may be able to diagnose UV damage. UV damage may be difficult to assess from a conventional superficial view as UV damage may be present even in wrinkle-free skin. However, UV damage may be assessed by examining skin structures for an increase in melanin production; global distribution, damage and count of superficial blood vessels; change in hemoglobin count: changes in the thickness of the epidermis; changes in the quantity and global distribution of collagen, and the like. In an embodiment, diagnosis may not require processing the border of the lesion, as it may not be a key factor in final analysis of the skin lesion. In an embodiment, the algorithm 150 may be able to diagnose oral cancer.

In an embodiment, an algorithm 150 for determining a skin state 158 may enable cosmetics manufacturing validation or cutaneous clinical trials. For example, a skin state 158 may be determined prior to medical, non-medical, skin care product or cosmetics application and a time lapse series of images may be acquired to track the medical, non-medical, skin care product, and cosmetics effectiveness.

In an embodiment, there may be methods for storing, handling, integrating, and analyzing a skin state 158. The skin state 158 may be stored in the device 108 itself, on a PC, in a central server, a salon record, an e-medicine record, a medical repository, a cosmetic clinical studies database 115, a mobile device, and the like. The device 108 may communicate with a user interface 102, an online platform 120, a mobile platform 124, and the like to upload, deliver, share, and/or port images, analysis 154, skin states 158, user profiles, and the like. For example, a user may use a device 108 embodied in a cellular phone to capture an image of the skin and upload it to a mobile platform 124 for analysis 154 to determine a skin state 158. In response, the user may receive a personalized regimen 118 for sun protection given the user's skin state 158. Other factors that may be used to determine the regimen 118 may be the current UV Index, time of day, location, kind of sun protection product the user prefers, and the like. In the same example, the user may have already obtained a skin state 158 determination and they need not upload a new image but simply request a regimen 118 recommendation from the mobile platform 124 given the already determined and stored skin state 158. Once a skin state 158 is determined, it may be accessible by and/or integrated with any element of the user interface 102, online platform 120, mobile platform 124 and the like. A user may choose to share the skin state 158 as part of a practitioner record 180.

In an embodiment, an algorithm 150 for determining a skin state 158 may enable an analysis of differences and similarities among peers. The algorithm 150 may determine peers of a user who may be most like them in terms of skin state 158 or other criteria such as gender, age, ethnicity, behaviors such as smoking, working outdoors, and the like, diet, regimen 118, and any other identifying factors. The algorithm 150 may be able to interface with an online platform 120, third party database 115, or third party service provider 111 to access skin states 158 and demographic information for comparison. For example, a user may wish to know what other women in their mid-30's of the same skin color are using for foundation. By employing the algorithm 150, a user may be able to determine their own skin color, identify peers according to the search criteria, and view details on their peers' regimen 118 or the results of the specific search query 103. The algorithm 150 may enable grading of the skin relative to a peer group. Using the algorithm 150, a user's skin state 158 may be compared to a previously defined skin state 158 in order to monitor the skin state 158 over time. A user's skin state 158 may also be compared to the skin state 158 of other individuals or groups of individuals to identify peers whose skin state 158 is closest to the user. Once a peer, such as a similar individual or group, is identified, the system may display the skin care products and/or skin care regimen that is effective for the peer. Similarly, any comparison among users may be made by the system, such as a comparison of at least one of age, gender, location, climate, skin color, ethnicity, and the like, to identify a peer. In an embodiment, as the device 108 captures data from users and determines skin states 158, the information may be fed back into the algorithm 150 to further enhance the peer identification and product recommendation process.

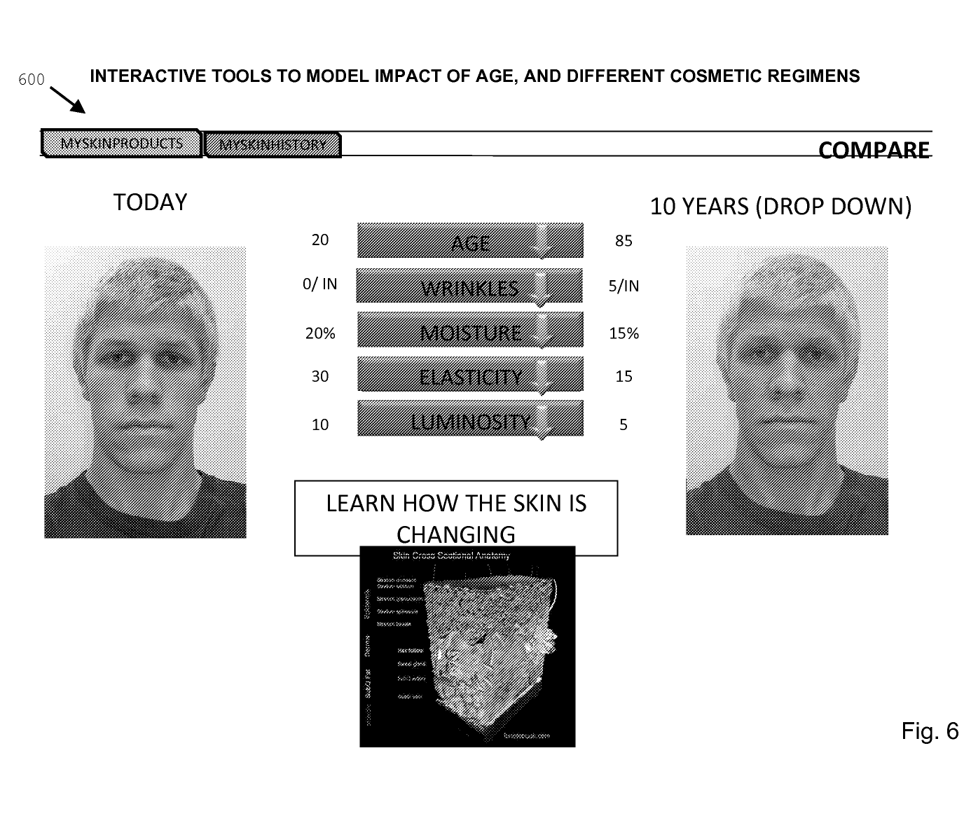

In an embodiment, an algorithm 150 for determining a skin state 158 may enable prediction/simulation tools 132. Having determined a skin state 158, an algorithm 150 may be able to simulate progression of aging, simulate skin care treatment effects and skin care and cosmetic regimens 118, simulate progression of a skin condition, and the like. Referring to FIG. 6, a user may use a user interface 102 to access the simulation tools 132. In the example, the image of an entire face may be used but it should be understood that simulation tools 132 may be used to generate simulations for any size area of concern. After selecting or capturing a starting image, a user may indicate the kind of simulation they would like to perform. For example, the user may like to perform a simulation of aging only, or a simulation of aging and treatment effects. The simulation tool 132 may return data on overall appearance, wrinkle count, elasticity, luminosity, moisture, product usage simulation, and the like. For example, the output may also include a split image with the original face on one half and a new simulated output on the other half.

In an embodiment, an algorithm 150 for determining a skin state 158 may enable skin cycle monitoring 140. By monitoring skin at determined intervals, skin conditions with a cyclical nature may be monitored, predicted, pre-empted and the like. For example, skin conditions associated with a season, weather, pollen count, hormone level, environmental condition and the like may be identified and monitored by a skin cycle monitor 140.

In an embodiment, an algorithm 150 may be used to generate searchable and/or indexable tags to associate with images and may take advantage of image tagging. Images may be tagged with information relating to the content of the image, such as information relating to a skin state, a skin condition, a gender, an ethnicity, an age, a regimen, a treatment, and the like. The information may be gathered by algorithmic analysis, user input, visual inspection of the image, and the like. An algorithm 150 may be used to perform a search 103 using the information associated with the image as a search term. In embodiments, the information may be stored separately from the image, such as an entry in a user profile, or may be stored in association with an image. In an embodiment, a search 103 may be performed against information or images from other users' or a third party database 115 to identify similarities or differences in images or information. For example, a user may use information to search for peers with a similar skin condition in order to determine what to expect as the condition progresses. In another embodiment, the search 103 or query for advice or recommendation from experts may be performed against product information 190, wellness information 192, skin care regimens 118, third party experts 105, and the like. For example, a user may use information to search for product information 190 indicating an effectiveness of a product for the user's skin condition. In an embodiment, the search 103 may be performed to determine an availability of a product, an inventory of a product, a price of a product, and the like. For example, a user may use the information to search a store catalog for a specific product that may be effective for the user. In the example, the user may be pale skinned and be interested in identifying an inventory of a self-tanning product formulated specifically for pale skin. In an embodiment, the image itself may be used as a search query 103. For example, the image itself may be used to search a database 115 of skin images. In an embodiment, images and information entered into the system 104 may be leveraged to develop new algorithms 150 for enhanced diagnosis. For example, algorithms 150 may be developed for non-skin specific diseases with dermal manifestations, such as rheumatoid arthritis.

In an embodiment, an algorithm 150 may be useful for analyzing product characteristics. For example, an algorithm 150 may be able to take product ingredients and match the product up with a projected effectiveness on a particular skin state 158.

In an embodiment, new algorithm 150 development by practitioners, users, service providers 111, and the like may be enabled by a software development kit that anyone could use to develop new algorithms 150 and APIs 154 for the device 108.

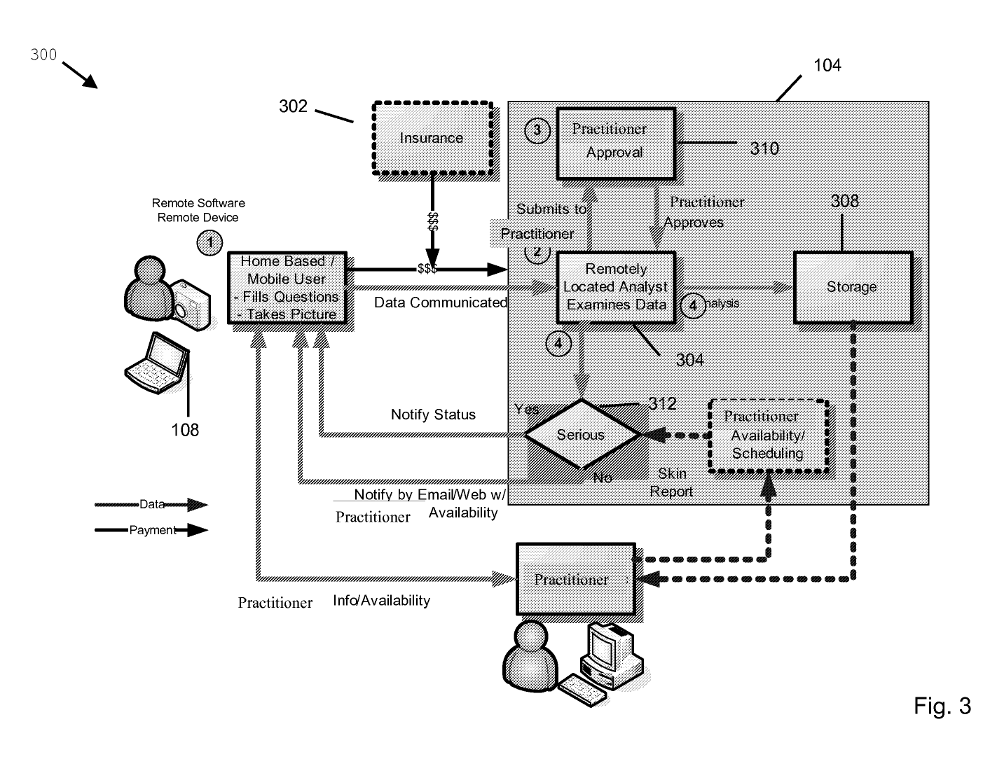

Referring now to FIG. 3, in an embodiment, a process for collecting images, performing skin analysis, communicating findings and scheduling follow up, if required may commence with image capture by a user using a device 108. The user may also answer questions or provide additional details regarding a user-entered imaging, cosmetic regimen, area of concern, or the like. Using the user interface 102, the data may be communicated to an analyst 304 or a computer for analysis 154 by any communication method, such as over a network, the Internet, wirelessly, and the like. In certain embodiments, as the data are collected or communicated, a payment system 302 may be accessed by the user. In the example shown, an insurance company may access the data, however, payment may be effected or requested by any interested entity such as a one-time payment by the user, a subscription by the user, a third party service provider 111, a platform 120,124, a practitioner, and the like. The entered data may be analyzed by the analyst, by software in real-time, by analysts assisted by software assistance, and the like. An initial analysis may be to determine data integrity. In instances where the data do not pass the integrity test, it may be communicated back to the user. The analyst's assessment may be assisted by software that uses an algorithm to determine type of condition and/or recommended care/treatment. Historical analysis and data, and modeling tools may be used to assist the analyst's assessment. Relevant parties (company personnel, payment providers, physicians, medical personnel, users, amongst others) may receive the analysis and/or user specific details for follow up or other actions that may be required. The analysis 154 may be stored 308 by the system and/or submitted to a practitioner for approval 310. In embodiments, storage 308 may require practitioner approval 310. A test of the severity 312 may determine the selection of an appropriate method of communication with the user. If the result of the test 312 is positive, the user may be notified immediately by a preferred communication method, such as telephone, instant message, and the like. If the result of the test 312 is negative, the user may similarly be notified, however, the notification may take a less urgent route, such as by email or postal mail. In any event, the software tool may recommend an appropriate communication method and media, based on the assessment and may populate preset templates with the information/message to be communicated. In addition, notification by any means may also include a notification of practitioner availability. The analysis 154 may trigger a practitioner availability/scheduling tool. For example, prior to transmitting the results on severity 312 to the user, a practitioner availability may be assessed and transmitted simultaneously. The user may access availability and scheduling tools in order to obtain and confirm an appointment time.

In an embodiment, a user interface 102 for a skin analysis system 104 may be used to interface with the device 108, store images, deploy algorithms 150, track a skin state 158 by keeping track of images from any number of areas of concern, the interval between image capture, a projected next image capture date, communicate findings to a practitioner, interact with simulation tools 132, skin type determination tools 130, a skin cycle monitor 140, practitioner availability/scheduling tools, and the like.

In embodiments, the user interface 102 may be operable as an application running on a device 108, a computer, server, kiosk, or the like, on an online platform 120, on a mobile platform 124, and the like. Any and all aspects of the user interface 102 described herein may be applicable to the user interface 102 running in any environment.

In an embodiment, the user interface 102 for the device, as will be further described herein, may be integral with the device 108, such as embodied in the keypad of a communications device or a series of buttons, switches, keys and the like disposed on the device 108, or may be external to the device 108, such as software running on a computer, on the Internet, on an intranet, on a mobile communications device, on an online platform 102, on a mobile platform 124, and the like. The user interface 102 may be used to modify a setting of the device 108, such as the magnification, light source, light intensity, wavelength of light, angle of light, electrical and magnetic properties of the light, positioning of sensor, duration of image capture, image size, data storage, data transmittal, and the like.

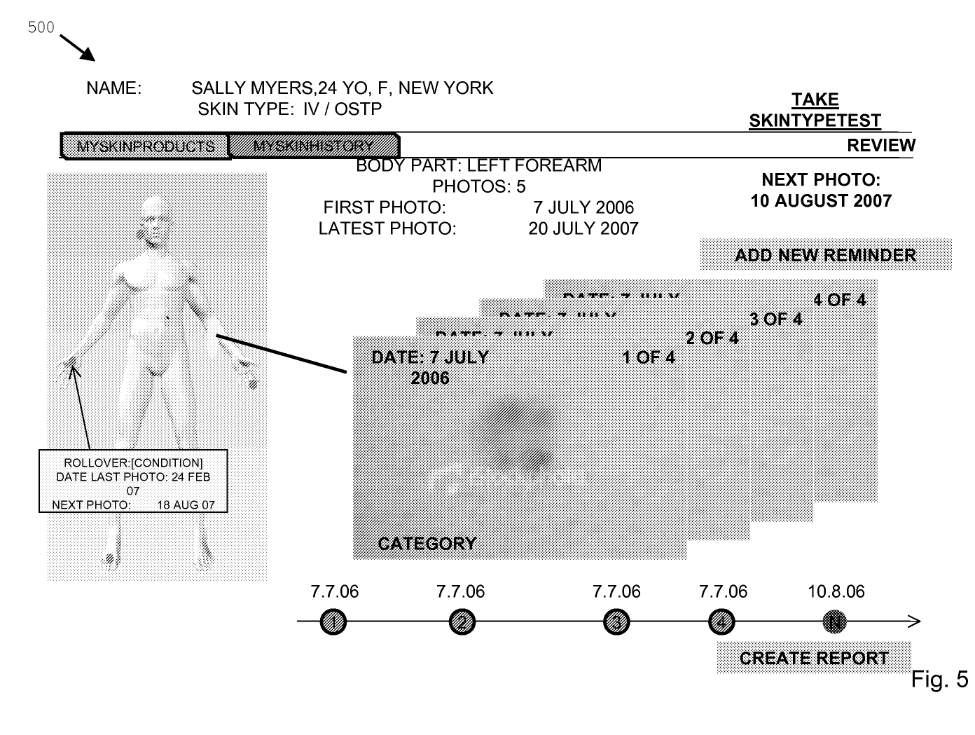

Referring now to FIG. 5, the user interface 102 may organize and index images captured by date, area of concern, skin state, and the like. For example and without limitation, as seen in the FIG. 5, four images captured from the same area of concern are indexed by their number within the series. In an embodiment, the user interface 102 may show in real time the field of view on the skin being imaged as well as populate the user interface 102 with the images once taken or once submitted by the user. The user interface 102 may keep track of the first image, latest image, next image, and the like. The user interface 102 may allow users to shuffle through image s and use the images as a basis for simulation 132, as described herein. The user interface 102 may be used to set a reminder for next image capture. The user interface 102 may be used to create a report of the images and skin state 158. The user interface 102 may be used to transmit the report to a practitioner. In an embodiment, the user interface 102 may be used to launch a skin type test. In an embodiment, the user interface 102 may depict a form of a body. As a user interacts with the depiction of the body, such as with an indicating device, the portions of the body that have been imaged may be linked with the images such that the images may pop-up or be otherwise accessed. The user interface 102 may be adapted to collect data from the user in response to prompts. The user interface 102 may employ an algorithm 150 to check the integrity of the captured images. The user interface 102 may guide the user in capturing images and providing user input in association with the images.

In an embodiment, the user interface 102 may interface with host hardware 108 or third party hardware 109. Hardware 108, 109 may comprise an imaging device that may connect with a computer, online platform 120, mobile platform 124, and the like via the user interface 102 and enable users to capture an image that enables measure various skin health, condition and type parameters. The hardware device 108,109 may be a standalone device or connect via or be embodied in a computing device of either medical or non-medical use. The user interface 102 may guide the connection process for the hardware device 108, 109. The device 108, 109 may store images, reports and recommendations generated and maintain a repository of the image, all as part of a skin health record 121. It may enable a systematic storing of the skin health record 121. Third party hardware 109 may comprise devices such as moisture sensors, cosmetic analysis machines, dermascopes, cameras, x-ray machines, MRIs, medical record providers and software, web cameras, communication devices, and the like. Third party hardware 109 may connect to the system 104 seamlessly to enable the user to gain a better analysis, and share such sets of data with other experts or users.

In an embodiment, the user interface 102 may enable type determination 130. Characteristics may be captured to determine the skin characteristics and the skin state 158 of the users' skin. Broad genetic parameters, such as ethnicity, skin color, location factors, environmental factors (such as pollen count, weather, etc.), and lifestyle factors may be collected in addition to image and skin health data to determine the users' skin state 158. This skin state 158 may be correlated with product experience ranking and ratings 138 to enable providing a recommendation for most effective products.

The user interface 102 may display a regimen 118. The regimen 118 may be a feature that enables users to learn what products and product usage pattern would work best for their skin based on a skin care assessment 160 and/or type determination 130 and product experience sharing via ranking and rating 138 and/or comments regarding product effectiveness and experience (such as smell, taste, feel, texture, color, and the like). The regimen 118 may be a dynamic recommendation based on users' collective inputs as well as experts' inputs on products that would best suit the user's individual needs.

In an embodiment, the user interface 102 may enable simulation tools 132. Users may be able to upload an image and model various skin parameters (such as moisture level in skin, collagen level, age, and the like.) and observe changes in the image. Additionally, users may be able to model the impact of various products and regimens 118 (skin care, cosmetic, medical, nail care, hair care, and the like) on the image. Simulation tools 132 may enable users to view changes on the entire image or split half of the image to show a comparison of modeled change with current image. The user's images could also be automatically or manually optimized for the best look and the products or regimen 118 to obtain that look may be provided. Simulation tools 132 may also enable consumers to model the skin characteristics or state 158 of other selected users or non-users, such as celebrities, luminaries, average users, and the like.

In an embodiment, the user interface 102 may enable a daily report 134. The daily report 134 may be a report that provides the user information largely customized and most relevant to the user based on their skin state 158. The daily report 134 may list skin care regimen 118 to be followed based on the environmental and lifestyle factors relevant to the user, may indicate new product information 190, show the current skin care shelf 114 and rankings 138 or change in rankings 138, feedback from users or experts 105 on products most relevant to the user, and the like. The daily report 134 may include information about clinical trials and upcoming results, new product releases and status, events, various factors affecting the skin such as the day's weather forecast, UV index, temperature, pollen count, and the like, and other data to provide value to the user. The daily report 134 may report on whether a product is nearing its shelf life or may require replenishment based on a recommended usage protocol. The daily report 134 may be provided to the user by the user interface 102, paper, email, SMS, RSS, video or any other communication media.

In an embodiment, the user interface 102 may enable a wishlist 134. The wishlist 134 may be a function that a user could select and add products so that other people could see the wish list 134. Other users could then select the products off the wish list 134 and purchase and send the product to the user.

In an embodiment, the user interface 102 may enable ranking and rating 138. Ranking and rating 138 may be performed for various product characteristics as well as on the various raters and rankers. Product experience may be collected from users in simple ranking and rating 138 format as well as textual comment data to be stored in a database. This ranking and rating 138 may be real time, and may be synthesized to show what is most relevant to the user based on like users or peers. In an embodiment, the user interface 102 may enable a skin cycle monitor 140. The skin cycle monitor 140 may indicate when the last image was collected and countdown to the next scan based on a time interval, such as the time required to replenish the skin or any other interval. Currently, it is believed that the skin replenishes itself every 28 days. The skin cycle monitor 140 may take into consideration age, environmental changes, and other factors to indicate the upcoming scan schedule.

In an embodiment, the user interface 102 may enable wellness/health 142. The user interface 102 may collect lifestyle data and also provide lifestyle (such as sleep, rest, exercise, and the like) and health (such as vitamins, food, products usage, and the like) recommendations based on the users particular skin state 158 and characteristics. The wellness and health module 142 may enable the user to obtain a personalized best fit health and wellness schedule and regimen 118.

In an embodiment, the user interface 102 may enable games 148. Users may be able to play games 148 that may enable users to model various products, try different hairstyles, model different hairstyles and clothes, and the like. Users may interact with other users or the computer to make the product selection a fun process. This process could also be used to collect information on user preferences and looks.

In an embodiment, the user interface 102 may enable a gift guide 144. Based on the user's skin state 158, personalized gift advice may be provided for the user.

In an embodiment, the user interface 102 may be embodied in touch screen user navigation. A touch screen system may be employed to enable the user to obtain a visual look and navigate to various parts of the user interface 102, such as navigate to the simulation tools 132, change picture orientation, drag and drop, and the like. Touch screen navigation may be particularly helpful as the hardware device 108 is connected to a computing platform. The user interface 102 may also enable collecting and coordinating information from other devices 109 and/or assessments, such as a dermascope, blood report, biopsy report, and the like to provide additional information for the skin record 121.

In an embodiment, the user interface 102 may enable a purchase/sample portal. The user interface 102 may include a purchase/sample portal that may enable the user to select products and complete a purchase or request a sample to be delivered to a pre-entered address. The portal may be available in various social networking platforms 188 as well as over various computing platforms, such as an online platform 120, mobile platform 124, computer, laptop, mobile phones, and other mobile devices, medical-use devices, and the like.

In an embodiment, the user interface 102 may enable scheduling and data sharing functionality. A user may be able to schedule online a meeting with a particular expert or practitioner and, if willing, then share a skin state 158 or specific parts of the skin record 121 and history in part or its entirety with the expert or practitioner. Ranked experts and practitioners, availability, and other criteria to aid the selection and scheduling process may be indicated to the user. Experts may also be able to share particular sets of data amongst themselves, such as among practitioners, physician to another physician, physician to spa, spa to spa, and the like.

Other inputs 112, such as devices, features and data, may be used to augment the data submitted by the user or as the primary data to obtain a personalized assessment regarding the users' beauty, cosmetic, or medical concerns related to skin, hair, nails, and the like. For example, certain devices may be available commercially off the shelf, purchased, proprietary, and the like.

In an embodiment, a wearable monitor 182 may be an input 112 to the system 104 and user interface 102. Wearable skin health monitors 182 may enable real time tracking of changes in the environment and the skins health. These devices could be worn directly on the body, or integrated into clothing, apparel and/or accessories carried by the user. An example would be a user having a device that monitors the UV level, and provides a warning if the sun protection level accorded by a product used by the user falls below a set target level. These wearable monitors 182 may have independent user interfaces 102 or can be programmed for personalized parameters using other input devices. Wearable monitors 182 may also capture various physical parameters like heart rate, blood pressure, exercise rate, water consumption, fat counter, calorie meter, and the like.

In an embodiment, product information 190 may be an input 112 to the system 104 and user interface 102. A database of product information 190 may comprise product, name, claims, manufacturer information, ranking and ratings 138, packaging information, images, usage parameters, product development history or forecast, special handling, upcoming changes, safety information, effectiveness information, smell, taste, color, texture, price, geography of manufacturing, brand information, consumer feedback and experiences, and other such parameters that may be obtained and/or maintained to assist in the selection of the best product suited to the users' individual preferences or conditions to obtain the best beauty or medical outcome for their skin, hair, nails, and the like. Additionally, similar information on service oriented products such as massages, facials, hair toning, and the like may also be captured as well as information on procedures such as liposuction, Botox treatments, laser hair removal and other beauty, cosmetic and/or medical procedures related to helping the user look good, improve or maintain a skin state 158, and the like. Manufacturers may register product information 190, contribute information on procedures, products in the pipeline, products in clinical trials, and the like. Users may rank and rate 138 products. A database update utility may update the database with new product information 190, store inventory, and the like.

In an embodiment, wellness information 192 may be an input 112 to the system 104 and user interface 102. Health and wellness information 192 may be captured, such as the impact of various products, primarily but not limited to non-prescription medications, supplements and other consumables that assist and maintain health and wellness (such as vitamins, protein shakes, supplements, and the like). Additionally, information on lifestyle recommendations (such as sleep, rest, diet and exercise recommendations for particular age groups/ethnicities, etc.) may be collected and correlated with user preferences and characteristics to enable and provide a holistic health, wellness, and beauty/cosmetic optimal personalized solution and service.

In an embodiment, a plug-in web capture 194 may be an input 112 to the system 104 and user interface 102. A software component-plug in for internet web browsers and basket or repository may recognize graphic objects on any browsed web page and allow the user to select, and drag-and-drop the graphic object onto a basket or repository onto a page of the web browser, such as a page comprising the skin care shelf 114. The graphic objects would be recognized through a standard reference table that would be accessed remotely or reside on the user's PC as part of the plug-in module 194, or as part of a resident software program on the computing platform. Graphic objects may include images for commercial products, such as skin care products or creams, or other objects that are part of any web e-commerce site. Once recognized, the plug-in 194 may highlight the picture, notifying the user that is it recognized, or provide additional information or reference. The plug-in 194 may also recognize brand names, trade names, generic pharmaceutical names, trademarks, and the like.

In an embodiment, barcode scan 198 may be an input 112 to the system 104 and user interface 102. Bar code information on various products may be captured to assist tracking, identification, price determination and correlation with other product information 190 for identifying similar substitute products, or other allied product information, usage recommendation, other user experience, pricing and delivery information, amongst other relevant sets of data. The bar code scanner 198 could be part of the hand held user device 108, a standalone system, a manual entry mechanism, and the like.

In an embodiment, conventional information/questionnaires 101 may be an input 112 to the system 104 and user interface 102. Information 101 on the users and products may be captured via dynamic and static questions. Information such as age, sex, location, personal lifestyle traits, smoking habits, sleep patterns, skin dryness/oiliness and moisture levels, product likes and dislikes, experiences with other products along parameters such as smell, taste, absorption, staining propensity, and the like may be captured in a fun manner using questions and answers, games and other interactive tools interspersed at various points of the users' interaction with the service product, system 104, or user interface 102. Information 101 may be captured directly form the user or via an intermediary, and augmented automatically via computer data population, as an output of an algorithm 150 or by experts based on their assessment. Information 101 may be obtained by quizzes, badge- and widget-based forms, on-the-fly, through adaptive, investigative questioning, and the like. Information 101 may be obtained through questionnaires, such as How often do you go shopping?, When do you shop for cosmetics?, Where do you typically go? Why that spot?, Who do you shop with? Why?, What do you ask your friends when asking for advice?, Where do you go for new products/information about cosmetics?, When do you have to go to a dept store, vs buying online?, When would you want to know something immediately from your friends?, What do you ask from your friends?, How do you choose a mobile phone?, What do you care about menus on a cell phone?, When do you get a new cell phone?, and the like.

In an embodiment, third party experts 105 may be an input 112 to the system 104 and user interface 102. The system 104 may connect various experts such as practitioners, physicians, medical experts, aestheticians, schedulers, product ingredient experts, cosmetologists, herbal, ayurvedic and homeopathic experts, health and wellness experts, media experts, photograph enhancement experts, and the like with users and one another. Users may be able to direct questions to such experts 105 who may be located at different places geographically over the system to obtain personalized advice. The experts 105 may be provided with users' data and characteristics collected and a record of the experts assessment may be retained in the record 121. The recommendation provided by the expert may be offered to the user for purchase/sample request, and the like. Experts may also be able to flag certain cases or sets of data for discussion or referrals within the expert community or with users.

In an embodiment, third party hardware 109 may be an input 112 to the system 104 and user interface 102. The system may connect with various third party hardware 109, such as existing imaging solutions, camera devices, computers, lighting systems, sports devices such as pedometers, and the like.

In an embodiment, third party service providers 111 may be an input 112 to the system 104 and user interface 102. Third party service providers 111 may be integrated into the system 104 to enable users to make the best personalized product or service selection for their hair, skin, nails, and the like for medical or cosmetic/beauty needs, and the like. Third party service providers 111 may include hospitals, physicians, spas, salons, aestheticians, beauticians, cosmetic counters, drug stores, cosmetics sales representatives and websites, ranking and rating services, product information databases, testing laboratories, magazines and information providers, insurance companies, social networking sites, health and wellness services, photograph enhancement services, and the like. For example, based on a skin concern, the scheduling system for a physician may be integrated and scheduling options offered online to users, while also connecting with insurance providers to confirm coverage with the user. In addition, pre-assessments on the condition, availability of historical medical and/or cosmetic products prescribed either over the counter or by medical prescription, and/or recommended services may be captured to make the selection process for the user convenient and easy.

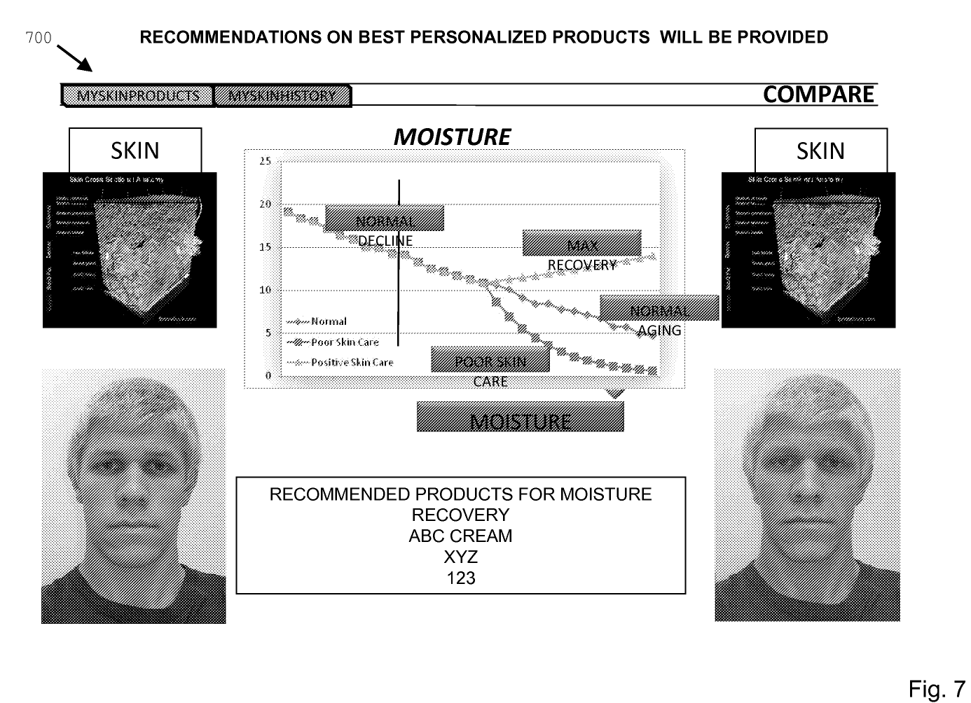

Referring to FIG. 7, a system for providing recommendations for skin care based on a skin state 158, a skin care goal, and environmental factors affecting the skin may comprise obtaining a skin state 158 of an individual, categorizing the individual by skin state 158, and recommending products and regimens that may be effective in achieving a skin care goal. The system may be computer-based, Internet based, network based, and the like. In an embodiment, the recommendation may be made on the basis of identifying other users with similar skin states and identifying a product or regimen that is effective for them. In an embodiment, the recommendation may be made on the basis of product information 190, wellness information 192, a third party database 115, an expert 105, a service provider 111, and the like. As seen in FIG. 7, a user may acquire an initial image and perform an analysis for a specific endpoint, such as moisture in this case. The system may automatically recommend certain products based on the moisture level that may be effective given the moisture level, a skin state 158, and the like. Additionally, the system may perform a projection of skin state 158 based on various skin care regimens 118, such as maximum care, normal care, or poor care. In an embodiment, the images may be captured using the device 108 or third party hardware 109. Images may be captured using any image capture device or technique, employing any kind of incident light, such as unpolarized light, polarized light, monochromatic light, diffuse light, white light, multiple single wavelength light, and the like. Any captured image may be used to obtain a skin state 158.

An embodiment of a skin care recommendation page of a skin care system may include a report of products the user is currently using, user input to obtain a skin state 158, a recommendation request, and the like. The report on the products the user is currently using may include ranking or ratings 138. For example, when a user accesses the user interface 102, they may access an adaptive questionnaire to determine their experience with their current regimen 118, current products or therapies used, or any products or regimens 118 used in the past. For example, the user may be asked to respond to questions such as How effective is it?, How is its fragrance?, How does it absorb?, Does it cause breakouts?, How does it feel?, Do you think this product is of good value?, and the like. Of course, rankings and ratings need not be prompted by questions but may simply be anecdotal, deployed in a non-question format, deployed in a drop down menu, and the like. To obtain a skin state 158, the user may enter data relating to aspects such as gender, age, ethnicity, location, skin color, environmental factors, and the like. In embodiments, analysis 154 of images obtained from the device 108 or third party hardware 109 may also be used to determine a skin state 158. Based on the skin state 158, either derived from user input, analysis of images, or a combination thereof, users may be able to determine products and regimens 118 that may work best for their skin state 158 by connecting to a database containing wellness 192, regimen 118, expert 105, service provider 111, and product information 190, wherein the information may comprise product ingredients, product claims, product indications, product pairing, product usage protocol, product ratings and rankings 138, and the like. By including rankings and ratings 138, recommendations may be made for skin related products adjusted for age, skin color, location, ethnicity, environmental factors, and the like. In an embodiment, the user may perform a recommendation request which may involve selecting a skin goal, such as moisturize, protect, cleanse, tone, beautify, anti-aging, wrinkle protection, skin tightening, deep cleanse, pore diminishing, treat rosacea, exfoliate, lighten skin, tan, sun protect, self-tan, treat acne, avoid pimples, improve luminosity, skin rejuvenation, treat spots, treat Crow's feet, hair removal, scar treatment, and the like. In embodiments, a skin goal may be automatically selected by the system 104. Automatic selection may be based on an aspect of the skin state 158. For example, if analysis 154 reveals that the skin is severely dry, the system may recommend moisturizing products for severely dry skin, or the system may recommend ingredients to look for in a product. The user may be able to purchase products directly from the recommendations page, such as by placing the product in an electronic shopping cart 113, or may be directed to another site for purchase. In an embodiment, the user may be able to obtain samples of recommended or non-recommended products directly from the recommendations page. The shopping cart 113 may be a functionality that integrates with the skin care shelf 114. Users may be able to use the personalized recommendations and select products either for purchase, or for sample delivery. The user may be prompted for personal information such as address, shipping method, credit card number and the like, and that information may be retained by the shopping cart 113.

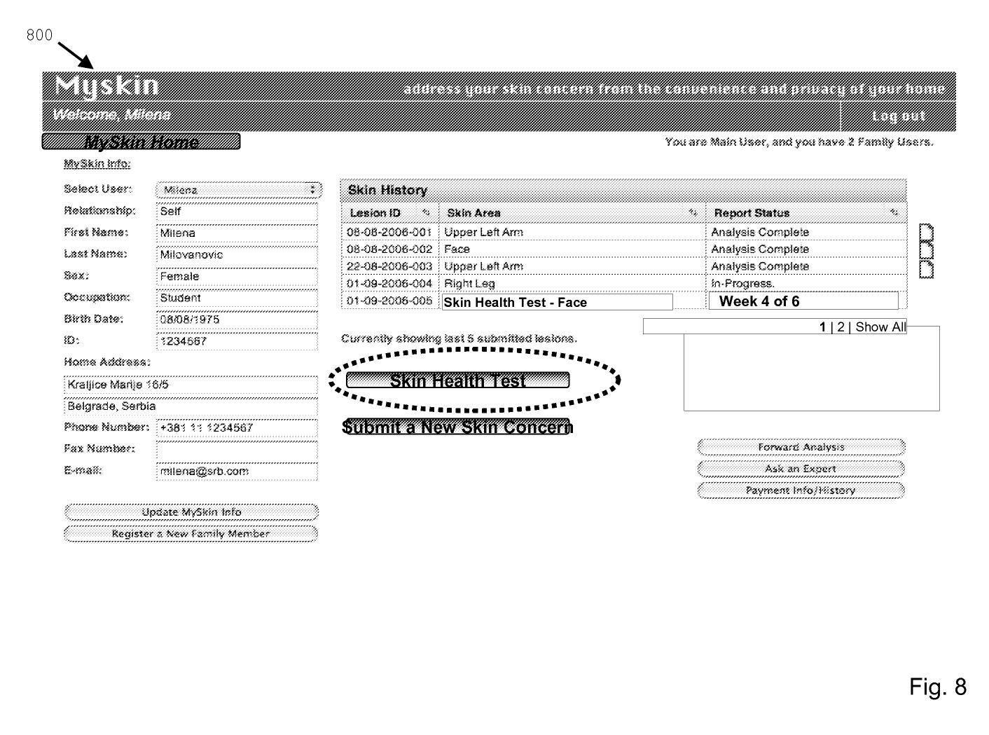

Referring to FIG. 8, a user interface 102 home page 800 of a skin care system 104 is depicted. The user may be prompted to input demographic information such as name, gender, age, occupation, ID, address, telephone number, email address, payment information, new related users, and the like, which may be stored in a user profile or as part of a skin record 121. The home page may show a skin record 121, or a listing of areas imaged, date imaged, and status of analysis. Once a task is complete in the skin history/record 121, an icon may be displayed near the Status. The user may be able to launch a new Skin Health Test from the home page 800 or submit a new skin concern. The user may be able to forward the analysis 154 to an interested party; Ask an Expert a question regarding an aspect of the skin, skin history/record 121, image analysis, and the like; view payment information and history; and the like.

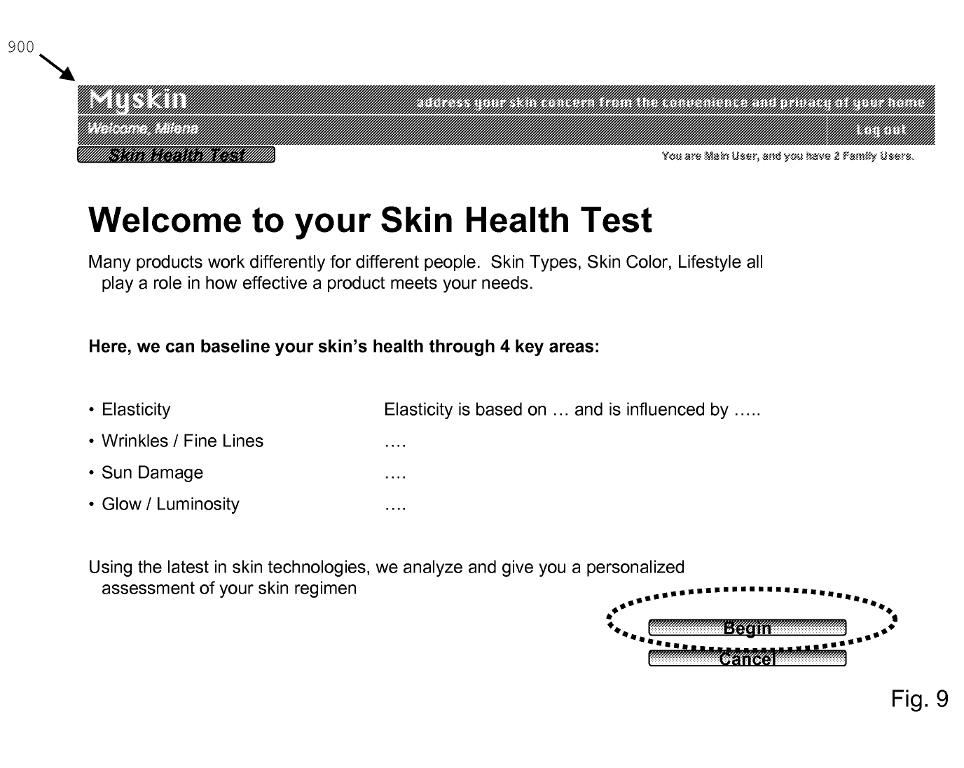

Referring to FIG. 9, a welcome page 900 of a skin health test is depicted. The welcome page may provide information on the skin health test, what endpoints will be tested for, such as elasticity, wrinkles/fine lines, sun damage, glow/luminosity, and the like. Using the analysis of the skin health test, the system may provide a personalized assessment of the user's skin regimen 118. The user may initiate the skin health test from the welcome page 900.

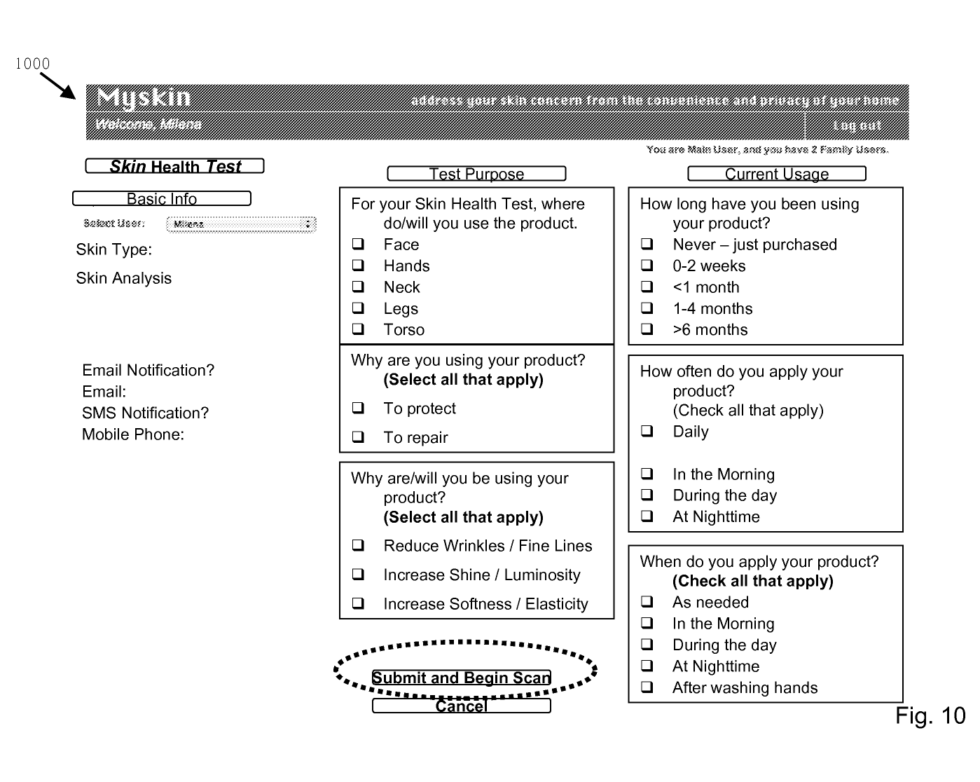

Referring to FIG. 10, a questionnaire page 1000 of a skin care system is depicted. The questionnaire may capture relevant skin history that may be useful for subsequent image analysis. The questions may be asked in multiple choice fashion or as open-ended questions. For example, a question may be ‘Where do you use your product?’ with responses including face, hands, neck, legs, torso, and the like. Another question may be ‘Why are you using your product?’ with responses including to protect, repair, moisturize, and any other skin care goal. Another question may be, ‘Why are/will you be using your product?’ with responses including reduce wrinkles/fine lines, increase shine/luminosity, increase softness/elasticity, and any other skin care goal. Other questions may include, ‘How long have you been using your product?’, ‘How often do you apply your product?’, ‘When do you apply your product?’, and the like, with responses including stated intervals of time. Other information gathered may be how the user prefers notification, where products were purchased, if the user employs a seasonal usage of products, and the like. From the questionnaire page 1000, the user may launch the skin health test.

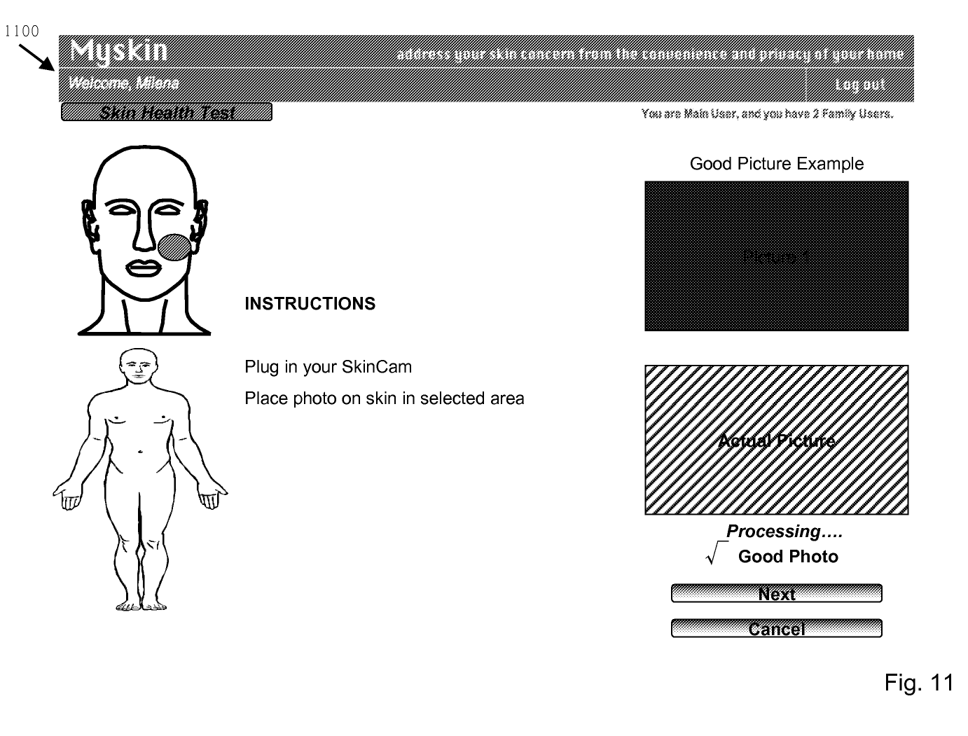

Referring to FIG. 11, a skin image capture page 1100 of a skin care system is depicted. In the example, the user interface 102 may access a device 108 in order to capture images, however, it should be understood that other devices 109 may be conveniently used in the system. The page 1100 may show a real time view of the area being imaged. The user may be able to employ positioning tools to be able to take an exact image of an area previously imaged. Once an image has been captured and submitted, an algorithm 150 may verify the integrity of the image. Once an image suitable for analysis has been captured, the user may proceed to an analysis page 1200.

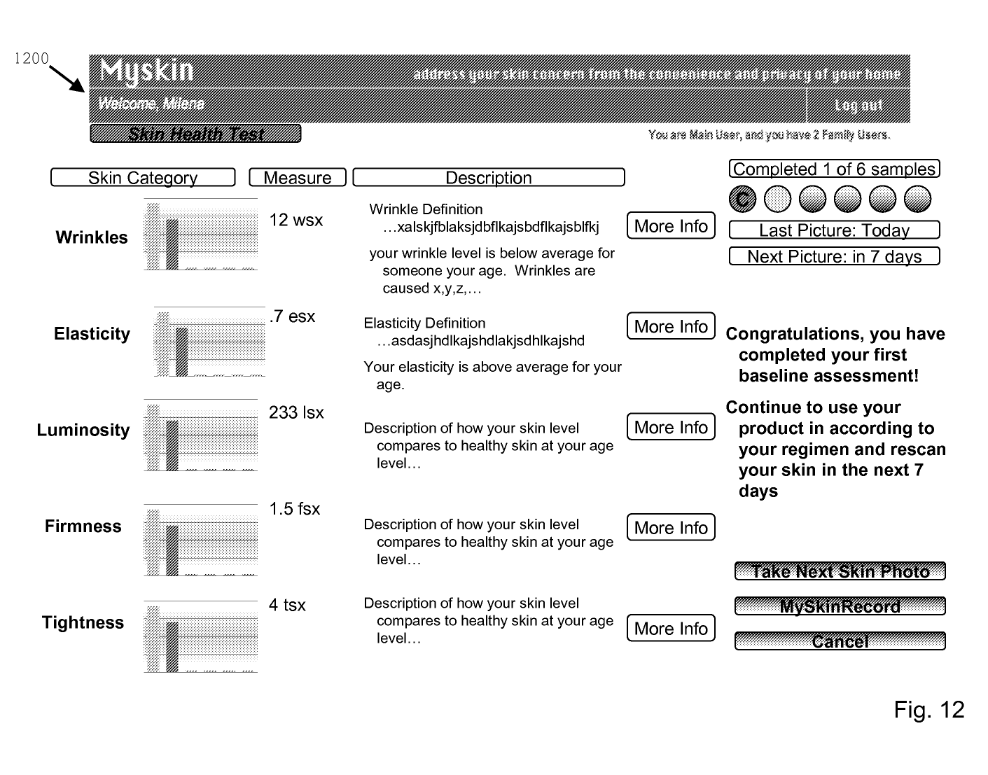

Referring to FIG. 12, a results page of a skin care system with bar graphs is depicted. Algorithms 150 may be used to analyze the image and provide measurements of wrinkles, elasticity, luminosity, firmness, tightness, and the like, as described previously herein. In an embodiment, the measurements may be quantitative measurements. The first analysis may be considered a baseline for purposes of tracking. For each measure, the user may be compared against the baseline for their age, skin state, gender, ethnicity, or any other category. For example, the graph depicts the reading for the user in the first bar on each graph and the average baseline for people of the same age in the second bar. It is apparent from visual inspection that the user is better than average, in this case. These results may be color-coded for ease of interpretation. The results page 1400 may include a description of each measure. The user may be able to request More Information for each of the measures, such as why a certain condition is caused and hints and tips on how to improve a skin condition. The user may be given instructions on when to re-scan the area, which products to use, which regimen 118 to employ, and the like. Desired improvements may be correlated to ingredients and most effective products for the user's skin may be recommended. The user may access and/or edit a skin record 121, which may contain information about the user, images, a chronology of images, information derived from the images, recommendations, products, regimen 118, and the like. The user may access a report facility to obtain a report.

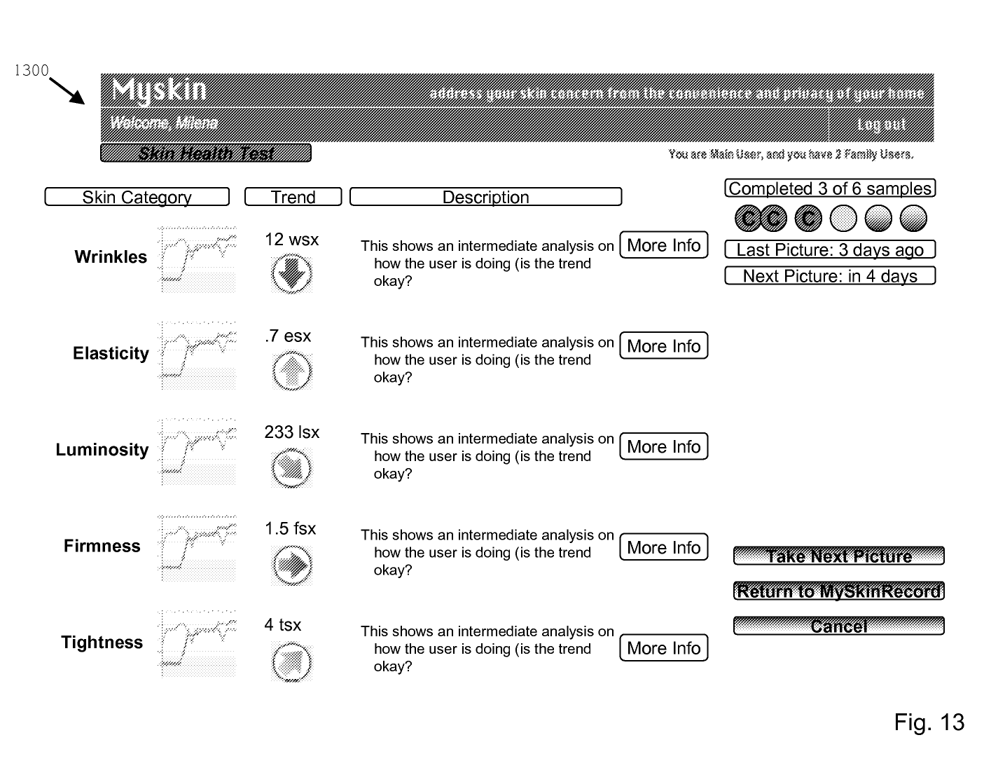

Referring to FIG. 13, a results page of a skin care system with trend analysis is depicted. A method for tracking the effectiveness of a skin care product or regimen may comprise obtaining a baseline skin health assessment; recommending a monitoring interval based on at least one of the skin care goal, product, and regimen; obtaining a second skin health assessment; comparing the second assessment to the baseline assessment to determine progress towards a skin care goal; and, optionally, optimizing the regimen 118 or product in order to improve a skin health assessment. When a subsequent image is acquired and submitted to the system 104, a trend analysis may be performed. Subsequent images may be used to track effectiveness of products and/or regimens 118 and, ultimately, advise the user on and optimize their skin regimen 118, product and/or condition. The trend analysis may be useful for determining an intermediate skin state 158 during a regimen 118. Progress may be shown over time. A time series of images, such as over a twenty-eight day skin cycle, over a treatment timeframe, seasonally, periodically over a year and the like may be captured in order to track progress of a skin state 158. The data may be presented in a pictorial view with data on the picture, graphical view, trend view, numerical view, text view, and the like. Progress may be sorted by the concerns/skin care goals that the user may have indicated at the beginning of the test. The user may be told when to take the next image, how much longer to continue with a regimen 118, how to modify the regimen 118, be reassured about the effectiveness of a product or regimen 118, receive useful tips, and the like. The user may view and/or edit a skin record 121. The user may be able to view past images and perform a simulation 132 of future progress. The user may access a report facility to obtain a report.

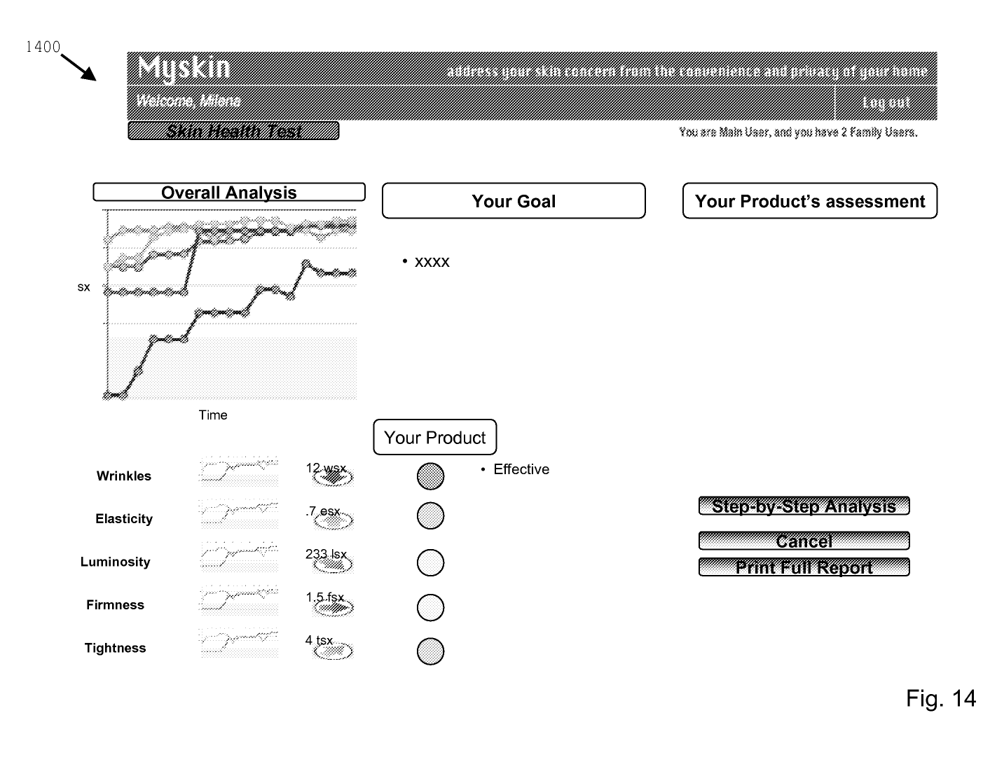

Referring to FIG. 14, a summary screen of a skin care system is depicted. An overall analysis for a time interval may be shown, current measurements, progress towards reaching a skin care goal, a product assessment, a regimen 118 assessment, advice on continuing, modifying, or terminating a regimen 118 or product usage, and the like. The user may view a step-by-step analysis or obtain a full report. At an interval, such as at the end of a suggested regimen 118, a report may include information on how the user's skin state 158 changed over time, if the user's skin is healthier than when they started the regimen 118, if the product or regimen 118 met their initial goals, feedback on regimen 118/product effectiveness, and the like. Given the current skin state 158, a new product or regimen 118 may be recommended. For example, the system may recommend specific ingredients to look for in order to increase a user's luminosity given a current skin state 158. Reports may be on-screen, printed, custom, and the like. Reports may be shared with a practitioner for ongoing treatment and consultation.

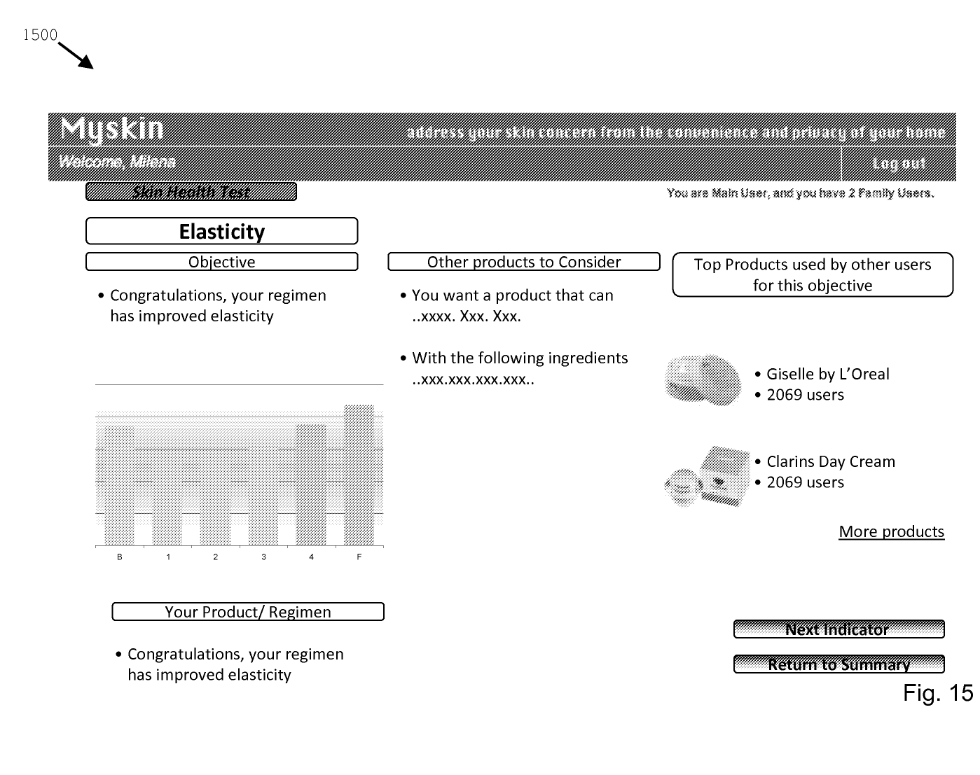

Referring to FIG. 15, an elasticity summary page 1500 of a skin care system is depicted. A step-by-step analysis of each indicator may be performed. For example, a step-by-step analysis of the elasticity measurement is shown in FIG. 17. The summary page 1500 may depict all of the data captured over an interval, such as in a bar graph, for each indicator on separate summary pages 1500. Progress towards meeting a skin care goal may be indicated by the data and its analysis or from user input. An assessment of a user's product or regimen 118 in meeting the skin care goal may be made. Products or regimens 118 that may enable meeting future needs may be indicated. The system may also indicate products used or regimens 118 employed by other users in meeting the stated skin care goal.

In embodiments, a system for providing recommendations for skin care based on a skin state 158, a skin care goal, and environmental factors affecting the skin may comprise interaction with tools and algorithms 150 on an online platform 120, a mobile platform 124, a social networking interface, and the like to receive product and regimen recommendations and track product and regimen 118 effectiveness. The user interface 102 may reside on an online platform 120, mobile platform 124, or social networking interface and guide the user while also serving as a data repository to maintain a skin record 121 and history tracking tool, and may help the user organize information relevant to their condition in a logical fashion.

1. A non-invasive imaging device, comprising:
an illumination source comprising an incident light source to direct light upon skin; and
a detector for detecting the degree of polarization of light reflected from the skin.

2. The device of claim 1, wherein the illumination source is positioned to direct light at a selected angle alpha.

3. The device of claim 2, wherein varying alpha varies the depth of the measurement of the layers in the skin.

4. The device of claim 3, wherein each depth has a specific angle which produces a full polarized reflection.

5. The device of claim 1, wherein the incident light source is an unpolarized light source.

6. The device of claim 1, wherein the incident light source is a polarized light source.

7. (canceled)

8. The device of claim 1, further comprising a sensor for capturing an image of the reflected light.

9. The device of claim 1, further comprising an optical facility for detecting reflected light from the skin.

10. The device of claim 1, further comprising a communication facility for transmitting the detected information.

11. The device of claim 1, further comprising a storage facility for storing information collected by the device.

12. A method of determining a skin state, comprising:
illuminating skin with an incident light source;
detecting the degree of polarization of light reflected from the skin; and
determining a skin state based on an aspect of the polarization of the reflected light.

13. The method of claim 12, wherein the incident light is directed at a selected angle alpha.

14. The method of claim 13, wherein varying alpha varies the depth of the measurement of the layers in the skin.

15. The method of claim 14, wherein each depth has a specific angle which produces a full polarized reflection.

16. The method of claim 12, wherein the incident light is unpolarized light.

17. (canceled)

18. The method of claim 12, wherein the incident light is polarized light.

19. The method of claim 12, wherein the aspect of the polarization is at least one of an orientation, an amplitude, a phase, an angle, a shape, a degree, and an amount.

20. The method of claim 12, wherein determining is done using an algorithm.

21. The method of claim 20 wherein the algorithm involves artificial neural networks.

22. The method of claim 20, wherein the algorithm uses fuzzy logic.

23. The method of claim 20 wherein the algorithm involves fractal and multi-fractal analysis.

24. The method of claim 12, further comprising, filtering the reflected light to obtain polarized light of a wavelength defined by the filter output.

25. The method of claim 24, wherein algorithmic analysis is performed on the filtered image.

26. The method of claim 12, wherein determining involves creating an image of the difference between reflected diffusion light and reflected polarized light.

27. The method of claim 12, wherein determining involves comparing the aspect of the polarization of the reflected light to a calibration signal.

28-127. (canceled)
